(19) United States
(12) Patent Application Publication
(10) Pub. No.: US 2010/0029509 A1
(43) Pub. Date: Feb. 4, 2010

(54) NOVEL METHODS

(75) Inventors: Wei Huang, Sheffield (GB); Andrew Whiteley, Oxford (GB)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: NATURAL ENVIRONMENT RESEARCH COUNCIL, Swindon, Wiltshire (GB)

(21) Appl. No.: 12/513,953

(22) PCT Filed: Nov. 7, 2007
(86) PCT No.: PCT/GB07/04254

§ 371 Date: Aug. 17, 2009

(30) Foreign Application Priority Data
Nov. 7, 2006 (GB) 0622144.4

(51) Int. Cl. C40B 40/06; C12Q 1/68

(52) U.S. Cl. 506/16; 435/6

(57) ABSTRACT

The present invention relates to novel methods for producing a biosensor for detecting a specific compound, for identifying a gene encoding a regulatory protein responsive to a specific compound and for identifying a gene encoding a regulatory protein responsive to a specific compound.

The present invention relates to biosensors, and in particular to biosensors for detecting contaminant compounds, and to methods for producing biosensors. The invention also relates to novel methods for identifying genes encoding regulatory proteins and/or promoters responsive to particular chemicals.

Generally two types of biosensor are known (Belkin (2003) Current Opinion in Microbiol 6:206-212). The first type of biosensor couples a biological material with a microelectronic system or device to enable the rapid and accurate detection of specific compounds in a sample or environment, such as in body fluid, water or air. Biosensors of this type have typically relied on the specific interaction of enzymes and their substrates, or on the recognition between antibody and antigen, or on the accessibility of target molecules to their receptors, or on the high affinity of a nucleic acid strand for its complementary sequence.

The second type of biosensor, and the one of interest in this invention, uses a live, intact cell to detect a specific compound. This system allows the detection of very complex reactions which occur in the cell, and cannot easily be electronically mimicked. This type of biosensor also allows bioavailability and toxicity to be determined which cannot be reliably assayed using a microelectronic system. For example, using the first type of system it is possible to determine the amount of a compound in a sample, say of water or soil, however it is not possible to determine the amount of a compound which is bioavailable. Often if a compound is not bioavailable or in a form that is not toxic then its presence is not a problem, bioavailability and/or toxicity can only be reliably ascertained by using a live intact cell.

One area where the detection of bioavailable and/or toxic compounds is important is in assessing groundwater contamination or pollution and toxic chemical levels. In particular, to study compounds which have been identified to be among the list of “red lights” in the Organisation for Economic Co-operation and Development (OECD) Environment Outlook to the year 2020 (OECD, 2001). Common groundwater contaminating compounds include: aromatic solvents such as benzene, toluene, ethylbenzene and xylene isomers (BTEX), chlorinated compounds (e.g. trichloroethylene (TCE)), nitrates, and pesticides from agricultural runoff, such as, polycyclic aromatic hydrocarbons (PAHs; e.g. naphthalene, fluoranthene, pyrene) and polychlorinated biphenyls (PCBs). These are just some of a list of organic contaminants that are found in both aquatic and terrestrial ecosystems. These organic compounds are known to have varying degrees of toxic, mutagenic or carcinogenic activities.

While conventional analytical methods, such as mass spectrometry, gas chromotagraphy and high pressure liquid chromotagraphy can provide information about the concentrations of compounds in contaminated areas, they fail to indicate whether the compounds are accessible for assimilation by living organisms, that is, they fail to assess the bioavailability and toxicity of the compounds. An assessment of the bioavailability of a compound in the form in which it exists in nature is an important consideration for site remediation. Conventional analytical methods also require expensive equipment and highly trained technicians.

According to a first aspect, the present invention provides a method of producing a biosensor for detecting a specific compound comprising:
(1) cloning a gene which encodes a regulatory protein responsive to the specific compound into a first position in a first plasmid;
(2) cloning a promoter, which is activated in the presence of both the regulatory protein and the specific compound, into a second position in the first plasmid or into a second plasmid;
(3) integrating the cloned gene which encodes the regulatory protein, and the cloned promoter, into a chromosome of a host organism, wherein the promoter is operably linked to a means for detecting activation of the promoter.

Preferably the means for detecting activation of the promoter produces a detectable signal such as a visual signal, a smell, a taste or a machine detectable signal. The means for detecting activation of the promoter may be referred to as a reporter gene, and the two terms are used interchangeably.

Preferably the gene encoding the regulatory protein is heterologous to the host organism. Preferably the promoter is heterologous to the host organism. Preferably both the gene encoding the regulatory protein, and the promoter, are heterologous to the host organism.

In the context of this invention heterologous takes its normal meaning, that is, that the promoter and/or the regulator gene are from a different, but possibly related, species to the host organism.

The gene encoding the regulator protein and/or the promoter may be known sequences. Alternatively, the gene encoding the regulator protein and/or the promoter may be unknown sequences.

The term “responsive” refers to the fact that when the regulatory protein encoded by the cloned gene is in the presence of the specific compound it causes the activation of a specific promoter (typically the cloned promoter). Activation of the promoter may be achieved by the binding of the regulatory protein and/or the specific compound to the promoter. The specific compound may cause a conformational change in the structure of the regulatory protein which allows it to bind to and activate the promoter. Alternatively, activation of the promoter may occur via a cascade type reaction, which does not involve direct interaction between the regulatory protein and/or the specific compound and the promoter.

The cloned gene encoding the regulatory protein, and/or the cloned promoter, may be integrated into a chromosome of the host organism directly from one or more plasmid.

Alternatively, the cloned gene and/or the cloned promoter may be amplified from the plasmid by PCR, and then the PCR product may be cloned into a chromosome of the host organism.

Preferably the cloned gene encoding a regulatory protein, and the cloned promoter, are integrated into a chromosome of the host organism by homologous recombination.

Preferably the cloned gene encoding the regulatory protein, and the cloned promoter, are flanked by sequences homologous to regions in a chromosome of the host organism which allows recombination of the cloned gene and the cloned promoter into the chromosome of the host organism. Preferably the cloned gene and the cloned promoter are flanked by different sequences such that the cloned gene and the cloned promoter will integrate into the host chromosome at different positions.

The flanking sequences which allow homologous recombination may be very close to the cloned gene and/or the cloned promoter, that is, within a few base pairs, or the flanking sequences may be some distance away, for example, tens or hundreds of base pairs away. The further away the flanking sequences are from the cloned gene and/or the cloned promoter the more of the plasmid DNA that will be integrated into a chromosome of the host organism. In one embodiment, the plasmid may contain the cloned promoter operably linked to the means for detecting activation of the promoter (reporter gene), and the cloned promoter and the means for detecting activation of the promoter may then be recombined together into a chromosome in the host organism. In this embodiment the flanking sequences may be some distance apart, that is, at least the length of the promoter and means for detecting activation of the promoter apart.

In another embodiment the flanking sequences, for use in homologous recombination with a chromosome of the host organism, are introduced by PCR, and it is the PCR products which are integrated into a chromosome of the host organism.

The flanking sequences may be part of the Sal operon, provided the host organism has the Sal operon. The Sal operon allows an organism to metabolise salicylate. By using part of SalA as the flanking sequence for the gene encoding the regulatory protein, the gene can be integrated into a chromosome of the host organism. SalR is the regulatory protein of the Sal operon, and when expressed causes expression of SalA. SalR is constitutively expressed in organisms grown on salicylate. Thus, if the gene encoding the regulatory protein is cloned into the SalA gene of a host organism, and the host organism is then grown on salicylate, the SalR regulatory protein will be expressed and will cause expression of the SalA gene and/or the cloned gene encoding the regulatory protein which is cloned into the SalA gene.

By using homologous recombination to integrate the cloned gene and the cloned promoter into the host organism's chromosomal DNA the site of integration into the chromosome can be controlled. By controlling the site of integration the expression of the cloned DNA can be controlled. Also the disruption of genes essential to the host organism can be avoided.

As an alternative to homologous recombination the cloned gene and/or promoter may be incorporated into the host chromosome by illegitimate recombination. Preferably, the method of illegitimate recombination used is “homology facilitated illegitimate recombination” in which only one side of a piece of DNA to be integrated is homologous to the recipients's genome (Vries and Wackernagel 2002 PNAS vol 99 no 4 pg 2094-2099).

By locating the cloned gene which encodes the regulatory protein, and the cloned promoter, in a chromosome of the host organism the system is very stable. Previously, bacterial biosensors have used plasmid borne genes to facilitate the detection of compounds in a sample or environment, such biosensors require the plasmid to be retained by the bacteria. Retention of the plasmid requires selective pressure to be applied to the bacteria to ensure that the plasmid is retained; typically this is achieved by incorporating an antibiotic resistance gene in the plasmid and then including the antibiotic in the bacterial growth medium. This can be costly and if the bacterial biosensors are to be used in a sample, such as an environmental sample, may be difficult. If the plasmid is lost then the biosensor will not work, and may give false negatives if the user does not know the plasmid has been lost.

Preferably the compound detected by the biosensor is a contaminant, preferably an environmental contaminant.

The term contaminant includes any compound that may be viewed as contaminating or polluting a particular system. The system may be soil, ground water, any body of water, the air, a human or non-human body or body fluid, or any other suitable system.

The compound may be selected from the group comprising aromatic solvents such as benzene, toluene, ethylbenzene and xylene isomers (BTEX), chlorinated compounds (e.g. trichloroethylene (TCE)), nitrates, and pesticides from agricultural runoff, such as, polycyclic aromatic hydrocarbons (PAHs) such as naphthalene, fluoranthene, pyrene, and polychlorinated biphenyls (PCBs) and any other contaminant chemical compounds. Other contaminant chemical compounds include components of fuels, solvents, propellants, pesticides and any degradation products of these compounds.

Preferably the biosensor detects only bioavailable compounds.

The host organism may be any suitable competent host, that is any suitable host that can take up and recombine into its chromosome exogenous DNA. Preferably, the host organism is highly competent, and has a competence of more then 10⁻⁶. Preferably, the host organism displays a rate of integration of about 0.1%. By way of example, E. coli integrates at a rate of less than 0.001%, and is not a highly competent organism. Preferably the host organism is a bacterium or yeast. Preferably the host organism is naturally competent. Preferably the host organism is capable of recombination to introduce heterologous DNA into its chromosome. Preferably the host organism is able to tolerate the insertion of heterologous DNA, preferably the host organism is able to tolerate the insertion of greater than about 1 kb of heterologous DNA, preferably the insertion of more than about 2 kb can be tolerated, more preferably the insertion of greater then about 5 kb can be tolerated. Preferably the host organism is a bacterium of the Acinetobacter species or the Pseudomonas species, or any other gamma bacteria species.

Gamma bacteria have transport systems which allow the uptake of chemicals, and in particular aromatic chemicals, making them useful as biosensors for contaminant chemicals. More preferably the host organism is Acinetobacter baylyi.

The host organism may be naturally occurring or adapted by selective pressure/growth or may have been genetically modified.

Preferably the host organism is able to express the cloned genes. A problem encountered with some bacteria is that they are not always able to express heterologous DNA. For example, E. coli often has difficulties expressing genes from other bacteria, E. coli can not express dmpR, a phenol regulatory protein, originally from Pseudomonas sp. CF400.

Preferably the host organism is safe and easy to handle.

When the cloned promoter is integrated into a chromosome of the host organism the cloned promoter is operably linked to means for detecting activation of the promoter/a reporter gene. Operably linked means that the promoter and the reporter gene are arranged such that on activation of the promoter the reporter gene is expressed. Preferably, when the promoter is not activated there is no, or substantially no, expression of the reporter gene in the host organism. Preferably the reporter gene is operably linked to the promoter in the plasmid before integration into a chromosome of the host organism. Preferably, the reporter gene and the promoter are integrated into the host organism chromosome together. Alternatively, the reporter gene and the promoter may be integrated into a host chromosome separately; provided that once integrated they are operably linked.

Preferably the reporter gene when expressed due to activation of the cloned promoter produces a detectable signal. The detectable signal may be a change in enzyme function, metabolic function or gene expression. Preferably the amount of reporter gene expressed correlates with the amount of a specific compound which is bioavailable in a sample. Preferably expression of the reporter gene can be measured colormetrically or photometrically, for example by flourimetery. The reporter gene may express β-galactosidase which can be detected colormetrically. Alternatively, the reporter gene may be one or more of the firefly luciferase genes or the green fluorescent protein (GFP) gene, expression of which may be measured photometrically or flourimetrically. Preferably the reporter gene is one or more of luxA, luxB, luxC, luxD and luxE. Preferably the reporter gene is used without its natural promoter, with expression being driven by the cloned promoter.

Preferably the cloned gene which encodes the regulatory protein is constitutively expressed when integrated into the chromosome of the host organism. The gene may be constitutively expressed under all physiological conditions, or only under certain conditions, such as those used to test a sample. The promoter operably linked to the regulatory gene may be homologous or heterologous to the host organism.

Preferably a biosensor produced by the method of the invention works by the binding, or interaction, of the specific compound to be detected with the expression product of the cloned gene which encodes the regulatory protein. Preferably interaction between the expression product of the cloned gene which encodes the regulatory protein and the specific compound results in induction of the cloned promoter which results in the expression of the reporter gene. Preferably a complex is formed between the specific compound and the expression product (the regulatory protein) of the cloned gene which encodes the regulatory protein, and this complex activates/induces the cloned promoter and causes expression of the reporter gene.

Preferably the cloned promoter, and the cloned gene which encodes the regulatory protein, are both derived from an operon used by an organism to metabolise the specific compound which the biosensor will be used to monitor/detect.

Certain bacteria have evolved the capacity to use contaminant chemical compounds as food sources. Production of the required metabolic enzymes to utilise the contaminant chemical compound is often controlled by a particular type of regulatory protein that detects the contaminant chemical compounds through direct physical interaction. This protein-chemical complex then binds to a cognate promoter sequence and activates the expression of genes encoding the required metabolic enzymes. This type of regulatory protein, with its cognate promoter, can be used as a contaminant-detecting component in a biosensor of the invention. Suitable host organisms may be engineered such that the inducible promoter activated by the regulatory protein is operably linked to a reporter gene, such that interaction of the contaminant chemical compounds with the regulatory protein activates the promoter which drives expression of the reporter gene. Expression of the reporter gene provides a measurable signal which reflects the presence of the contaminant chemical compound, preferably there is a correlation between contaminant chemical compound level and the level of expression of the reporter gene.

For example, operons encoding genes required for the metabolism of contaminating chemical compounds such as phenol, toluene, benzene, napthalene and xylene are well understood and the regulatory protein, its corresponding gene and the inducible promoter from these operons could be adapted for use in the method and biosensor of this invention.

The regulatory protein and promoter used in the invention will depend on the target compound to be detected. For example, if the target compound is toluene or xylene, then the promoter may be the Pu promoter and the regulator gene may be xylR both derived from Pseudomonas putida. If the target compound is naphthalene the degradation operon, nahG, regulated by the nahR protein may be used (King J M H et al. (1990) Science 249 (4970): 778-781).

The cloned gene, which encodes the regulatory protein, and/or the cloned promoter may be naturally occurring, or may be derived from naturally occurring genes or may be synthetic. For example, the gene which encodes the regulatory protein, and/or the promoter, may be isolated from a bacterium which naturally metabolises the compound of interest. The gene and/or promoter may be used as they naturally occur, or they may be mutated or truncated. For example, the DNA sequence of the gene and/or promoter may be mutated to enable it to function or to improve its function in the host organism, or to improve integration into the host organism. Provided that the cloned gene, which encodes the regulatory gene, and the cloned promoter work together to allow the detection of a specific compound in the host organism, the sequence of the cloned gene and the cloned promoter does not matter.

A person skilled in the art will appreciate that any suitable plasmid(s) can be used in the method of the invention. The plasmid(s) must be able to integrate and retain the cloned gene and/or promoter. Preferably the plasmid(s) with the cloned gene and/or promoter can be replicated in a bacterial host in order to propagate the plasmid(s). The plasmid(s) can preferably also be taken up by competent host organisms, and be retained in the host organism whilst homologous recombination between the plasmid and a chromosome of the host organism occurs. The person skilled in the art will be able to include all appropriate regulatory sequences in the plasmid(s), such as, promoters, terminators, polyadenylation sequences, marker genes, flanking sequences for recombination, antibiotic selection genes and any other appropriate sequences. Examples of plasmids which can form the basis of plasmids for use in this method are the pGEM® plasmids available from Promega™ and the TOPO™ plasmids from Invitrogen Inc.

The method of the invention may be performed using one or two plasmids. If one plasmid is used, preferably the gene, encoding the regulatory protein, and the promoter are cloned into separate positions in the plasmid. This may be achieved by using different restriction enzymes. If two plasmids are used, preferably the gene encoding the regulatory protein, and the promoter, are cloned into different plasmids. In both cases, the gene encoding the regulatory protein, and the promoter are preferably flanked by different sequences which will allow their integration into different sites in a chromosome of the host organism.

Preferably biosensors produced by the method of the invention are capable of detecting nanomolar levels of a particular compound, making them as sensitive or more sensitive than conventional chromatography or spectrophotometry methods. Furthermore, chromosomal integration of (1) the gene which encodes the regulatory protein, (2) the promoter and (3) the reporter gene, produces a more sensitive system than a plasmid borne system as there is no or only very low background expression. In plasmid systems where there are multiple copies of the genes, and in particular multiple copies of the reporter gene, if the inducible promoter is even slightly ‘leaky’ then low level background expression of the reporter gene could give false positives.

Furthermore, because the number of plasmids in a bacterium can change then the results of different experiments can be difficult to quantitatively compare. The more copies of the plasmid the higher the level of detectable signal there is likely to be.

This method for producing a biosensor provides a convenient and effective approach to easily construct a biosensor for a specific substance as and when it is required. The present invention provides a method which enables the rapid creation of custom inducible bacterial biosensors, preferably in about 2-3 days (as opposed to months and the specialist genetics required previously).

It will be appreciated that all the preferred features of the invention discussed with reference the first aspect of the invention may be applied to all aspects of the invention.

According to another aspect, the invention provides a method of producing a biosensor for a specific compound comprising:
(1) identifying the specific compound:
(2) obtaining a pool of DNA;
(3) cloning fragments of DNA from the pool of DNA into a first and a second site in one or two plasmids;
(4) integrating the cloned DNA into a chromosome of a host organism, wherein the DNA from the first site in the plasmid is integrated into the chromosome at a first position such that it will be expressed in the host organism, and DNA from the second site in the plasmid is integrated into the chromosome at a second position such that the cloned DNA is operably linked to a reporter gene;
(5) applying the specific compound to the host organism; and
(6) screening for expression of the reporter gene.

Preferably expression of the reporter gene indicates that the host organism is responding to the presence of the specific compound and the organism can therefore be used as a biosensor for the specific compound.

According to a further aspect the invention provides a method of identifying (i) a gene encoding a regulatory protein responsive to a specific compound and (ii) a promoter activated by the regulatory protein and the specific compound:
(1) identifying the specific compound;
(2) obtaining a pool of DNA fragments;
(3) cloning fragments of DNA from the pool of DNA into a first and a second site in one or two plasmids such that DNA at the first site will be expressed when the plasmid is transformed into a host organism, and DNA at the second site is operably linked to a reporter gene;
(4) transforming a host organism with the one or two plasmids;
(5) applying the specific compound to the transformed host organism; and
(6) screening for expression of the reporter gene.

Expression of the reporter gene indicates that the host organism is expressing a regulatory gene, and has the associated promoter operably linked to a reporter gene, for the selected compound. The organism may be used a biosensor for the specific compound.

Alternatively, the gene and promoter identified by this method of the invention may then be integrated into the chromosome of a host organism to produce a biosensor for the specific compound.

In step 1 of a method of the invention the specific compound may be any compound of interest, in particular, the specific compound may be an environmental contaminant or pollutant. Examples of which are discussed above.

Preferably the pool of DNA used in the method of the invention is obtained by isolating DNA from a sample, such as a sample of soil, water, air or fluid. Preferably the sample is contaminated with the specific compound. Preferably the sample contains organisms that have evolved to survive in soil, water, air, fluid etc contaminated with the specific compound, and preferably some of the organisms have evolved to metabolise the specific compound. An aim being to isolate DNA from organisms which can metabolise the specific compounds which can be used in a biosensor.

Preferably the pool of DNA contains more than one different fragment of DNA. More preferably the pool of DNA contains 10 or more, 100 or more, 500 or more, 1000 or more different fragments of DNA.

Preferably the DNA used to produce the pool is isolated from an environmental sample without the culturing of the bacteria in the sample, this has the advantage that DNA from bacteria which are difficult or impossible to culture in a laboratory environment can be included in the pool. It is widely accepted that as many as 99% of bacteria are unculturable under laboratory conditions, the method of the invention ensures that the DNA of such bacteria is considered, and used when producing biosensors according to the invention.

By using a pool of DNA the method of the invention may be used to clone an unknown regulatory gene and/or promoter, or an unknown combination of a regulatory gene and a promoter from an environmental sample. No prior knowledge of the regulatory gene and/or the promoter responsive to a particular chemical to be detected by the biosensor is needed.

Preferably, a single pool of DNA may be used to screen for a regulatory gene and/or a promoter responsive to more than one chemical.

The DNA in the pool may be isolated as part of a total nucleic acid extraction process, or just the DNA may be extracted.

The pool of DNA used in a method of the invention may be digested with suitable restriction enzymes to allow it to be cloned into a plasmid. Suitable enzymes may include BglII and/or Sau3A.

Alternatively, the DNA may be cloned into a plasmid using blunt end ligation.

Preferably the DNA is inserted randomly into either the first or second site in the one or two plasmids. Both the first and second sites may be flanked by sequences that will allow homologous recombination of the cloned DNA into a chromosome of a host organism. The cloned DNA may be integrated into a chromosome of the host organism directly from the plasmid(s) or it may be amplified by PCR and the PCR fragment may be integrated into a chromosome of the host organism.

Alternatively, the cloned DNA may be amplified by PCR which uses primers to add flanking sequences to the cloned DNA which will allow homologous recombination into a chromosome of the host organism.

Preferably if the cloned sequences are integrated into a chromosome in the host organism they are integrated by homologous recombination.

The DNA integrated at the first position in the host chromosome, and/or the first site in the plasmid, is preferably arranged to be constitutively expressed, at least under test conditions. It is intended that this position of integration will allow genes encoding regulatory proteins to be trapped.

The DNA integrated at the second position in the host chromosome, and/or the second site in the plasmid, is preferably arranged to be located operably linked to a reporter gene. It is intended that this position of integration will allow promoter sequences to be trapped.

The reporter gene may be linked to the cloned DNA in the plasmid or on integration into a chromosome of the host organism.

Preferably, the method of the invention will allow the production of an organism into which there has been cloned a gene encoding a regulatory protein, and a promoter operably linked to a reporter gene, wherein the regulatory protein and the promoter work together in the presence of the specific compound to cause expression of the reporter gene.

By screening host organisms for expression of the reporter gene, and selecting only those that express the reporter gene in response to the specific compound, potential regulatory gene and promoter combinations, and thus biosensors, for the specific compound can be identified.

Preferably this method of the invention can be used to rapidly produce biosensors for chemical compounds where regulatory operons have not been isolated, or where only part of the operon has been isolated. The method of the invention will also allow genes involved in regulatory operons to be identified and cloned for further study.

This method of the invention has the advantage that is uses different sites to capture/trap promoter and regulatory sequences, rather than relying on one site to trap both. If the sequences are located a long way apart then a single site trap may not catch both, also if the sequences are orientated in opposite directions then a single site trap may not allow both sequences to function.

According to another aspect, the invention provides a method of identifying a gene encoding a regulatory protein responsive to a specific compound comprising:
(1) identifying a specific compound;
(2) obtaining a pool of DNA;
(3) cloning fragments of DNA from the pool of DNA into a plasmid;
(4) integrating the cloned DNA into the chromosome of a host organism such that the cloned DNA is expressed in the host organism, wherein the chromosome already carries a promoter operably linked to a reporter gene, and wherein the promoter is known to be activated in the presence of the specific compound and an unknown regulatory protein;
(5) applying the specific compound to the host organism; and
(6) screening for expression of the reporter gene.

According to a further aspect the invention provides a method of identifying a gene encoding a regulatory protein responsive to a specific compound comprising:
(1) identifying the specific compound;
(2) obtaining a pool of DNA fragments;
(3) cloning fragments of DNA from the pool of DNA into a plasmid such that the cloned DNA will be expressed when the plasmid is transformed into a host organism;
(4) transforming a host organism with the plasmid containing the cloned DNA, wherein the host organism carries a promoter operably linked to a reporter gene, and wherein the promoter is known to be activated in the presence of the specific compound and an unknown regulatory protein;
(5) applying the specific compound to the transformed host organism; and
(6) screening for expression of the reporter gene.

In both the preceding aspects of the invention, expression of the reporter gene is indicative that the host organism is carrying, either integrated into the host chromosome or on a plasmid, a gene encoding a regulatory protein responsive to a specific compound. Such organisms may be used as biosensors for the specific compound

Alternatively, the gene encoding a regulatory protein identified by a method of the invention, when the gene identified is on a plasmid, may be subsequently integrated into the chromosome of a host organism to produce a biosensor for the specific compound. The host organism must also have integrated into its chromosome a promoter operably linked to a reporter gene, wherein the promoter is activated in the presence of the regulatory protein and the specific compound, the host organism may then be used as a biosensor for the specific compound.

Preferably the cloned DNA is constitutively expressed, at least under test conditions.

By selecting host organisms that show an increase in reporter gene expression in the presence of the specific compound, a gene encoding a regulatory protein can be identified. This method therefore may serve as a method to trap genes which encode regulatory proteins and also as a way to produce a biosensor when the inducible promoter is known.

According to another aspect, the invention provides a method of identifying a promoter activated by a regulatory protein which is responsive to a specific compound comprising:
(1) identifying a specific compound;
(2) obtaining a pool of DNA;
(3) cloning fragments of DNA from the pool of DNA into a plasmid;
(4) integrating the cloned DNA into the chromosome of a host organism such that the cloned DNA is operably linked to a reporter gene, wherein the chromosome already carries a gene encoding a regulatory gene which is responsive to the specific compound;
(5) applying the specific compound to the host organism; and
(6) screening for expression of the reporter gene.

According to a further aspect the invention provides a method of identifying a promoter activated by a regulatory protein which is responsive to a specific compound comprising:
(1) identifying the specific compound;
(2) obtaining a pool of DNA fragments;
(3) cloning fragments of DNA from the pool of DNA into a plasmid such that the cloned DNA is operably linked to a reporter gene;
(4) transforming a host organism with the plasmid containing the cloned DNA wherein the host organism carriers a gene encoding a regulatory protein which is responsive to the specific compound;
(5) applying the specific compound to the transformed host organism; and
(6) screening for expression of the reporter gene.

In the preceding two aspects of the invention, expression of the reporter gene is indicative that the host organism is carrying, either integrated in the chromosome or on a plasmid, a promoter operably linked to a reporter which is activated in response to the specific compound and the regulatory protein. Organisms expressing the reporter may be used as biosensors for the specific compound.

Alternatively, a promoter carried on a plasmid identified by a method of the invention may be integrated into the chromosome of a host organism to produce a biosensor for the specific compound, provided that the host organism expresses the associated regulatory protein.

The gene encoding the regulatory protein must be expressed when the specific compound is applied. Preferably the gene encoding the regulatory protein is constitutively expressed in the host organism, at least under test conditions.

By selecting host organisms that show an increase in reporter gene expression in the presence of the specific compound, a promoter activated by the regulatory protein can be identified. This method therefore may serve as a method to trap genes which encode inducible promoters and also as a way to produce a biosensor when the gene encoding the regulatory protein is known.

According to another aspect, the invention provides a method of detecting in a sample the presence or absence of a particular compound comprising:
(i) contacting a biosensor made according to a method of the invention with the sample;
(ii) observing whether reporter gene expression is increased in the biosensor.

According to another aspect, the invention provides a kit for use in detecting a chemical compound in a sample, comprising a biosensor made according to the method of the invention and instructions to use the biosensor.

Preferably the biosensor is provided in a receptacle which minimises the chance of release of the biosensor into the environment.

Preferably the instructions to use the biosensor indicate how the biosensor and the sample should be mixed, and how to monitor expression of the reporter gene.

The kit may also include an indication of what concentration of chemical compound will give what level of reporter gene expression.

According to another aspect, the invention provides a kit for use in producing a biosensor comprising one or two plasmids with two cloning sites, one on each plasmid or two on one plasmid, and instructions to use the method of the invention to produce a biosensor.

The cloning sites may be multiple cloning sites.

The kit may also include a host organism.

The kit may include plasmids and/or a host organism as described with reference to any method of the invention.

The skilled man will appreciate that the preferred features referred to with reference to only one aspect of the invention may be applied to all aspects of the invention.

Preferred embodiments of the invention will now be described by way of example with reference to the accompanying drawings in which:

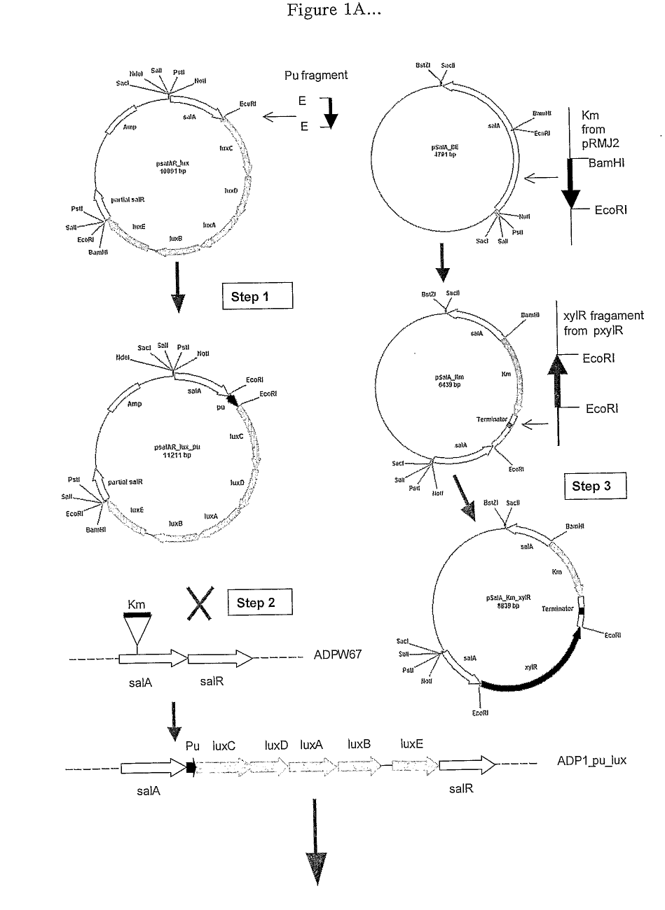

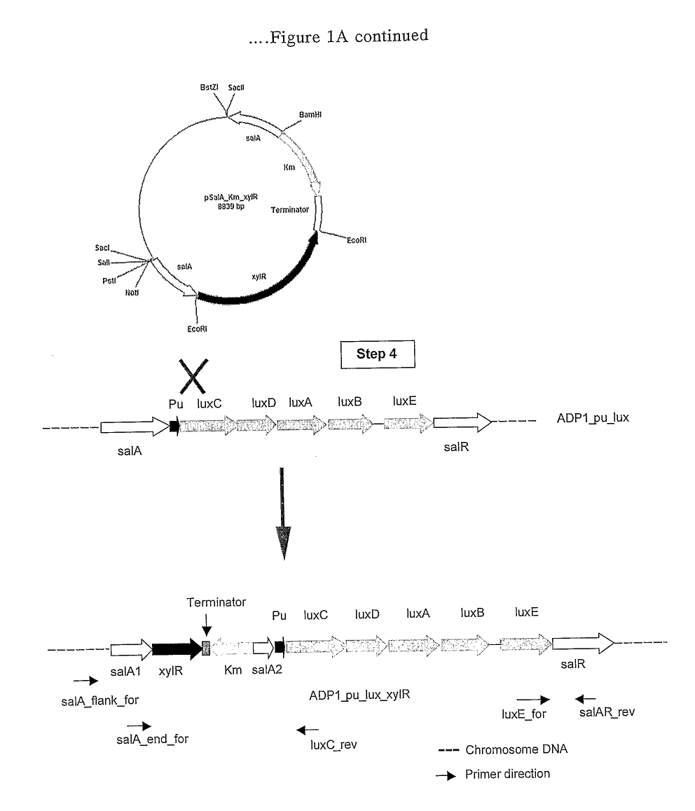

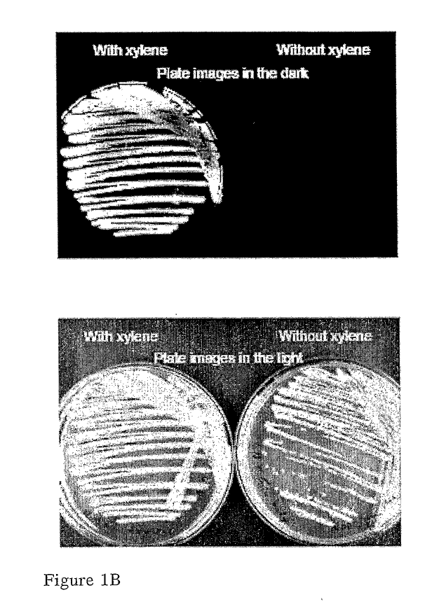

FIG. 1A depicts schematically the construction of Acinetobacter baylyi mutants ADP1_Pu_lux_xylR. More specifically, FIG. 1A shows the four steps taken to integrate the promoter Pu and the regulation gene xylR into the chromosome of Acinetobacter baylyi ADP1 to create the strain ADP1_Pu_lux_xylR.

FIG. 1B depicts ADP1_pu_lux_xylR growing on agar medium with or without the inducer m-xylene, the growth medium used is LB with 10 mM glucose.

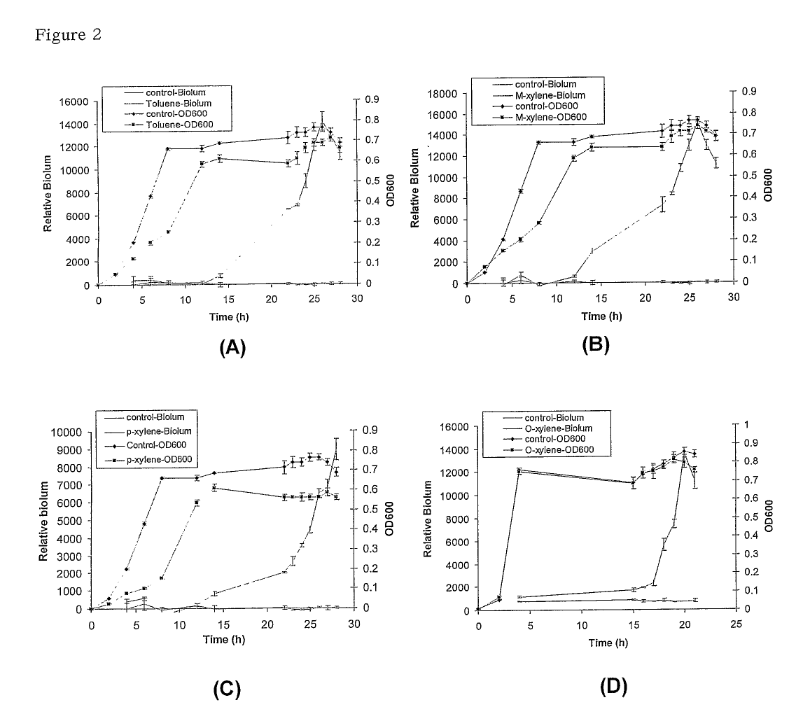

FIG. 2 illustrates growth-phase dependent xylR/Pu gene regulation in Acinetobacter baylyi ADP1_pu_lux_xylR. Toluene (A), m-xylene (B), p-xylene (C) or o-xylene (D) were added as inducers with final concentrations of 500 μM. The samples were incubated at 28° C. with shaking.

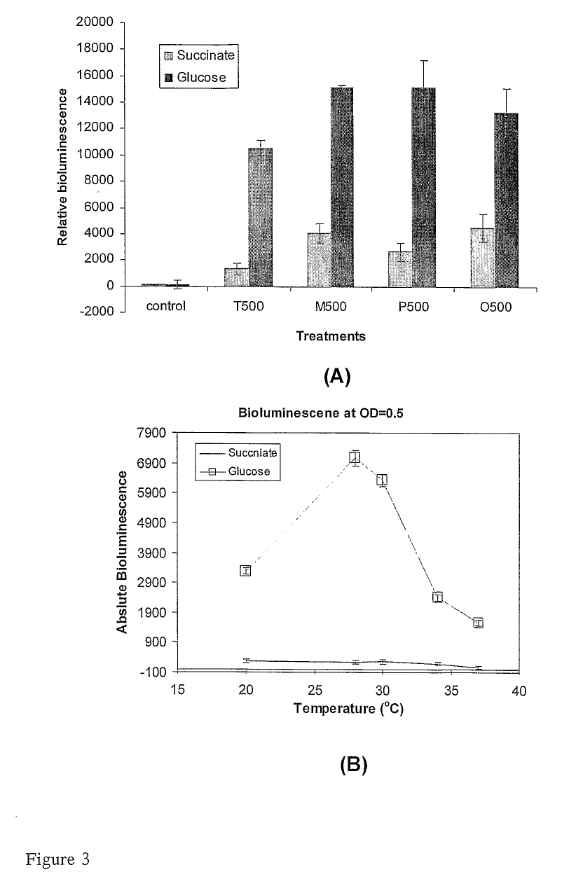

FIG. 3 illustrates the relative bioluminescence expression of ADP1_pu_lux_xylR in minimal medium supplemented with 50 mM glucose or 50 mM succinate as sole carbon source. FIG. 3A—Toluene (T), m-xylene(M), p-xylene(P) or o-xylene(O) were added separately as inducers with final concentrations of 500 μM. The samples were incubated at 28° C. with shaking. FIG. 3B—different temperature effects on Pu activity are illustrated. Absolute bioluminescence was measured at OD600=0.5.

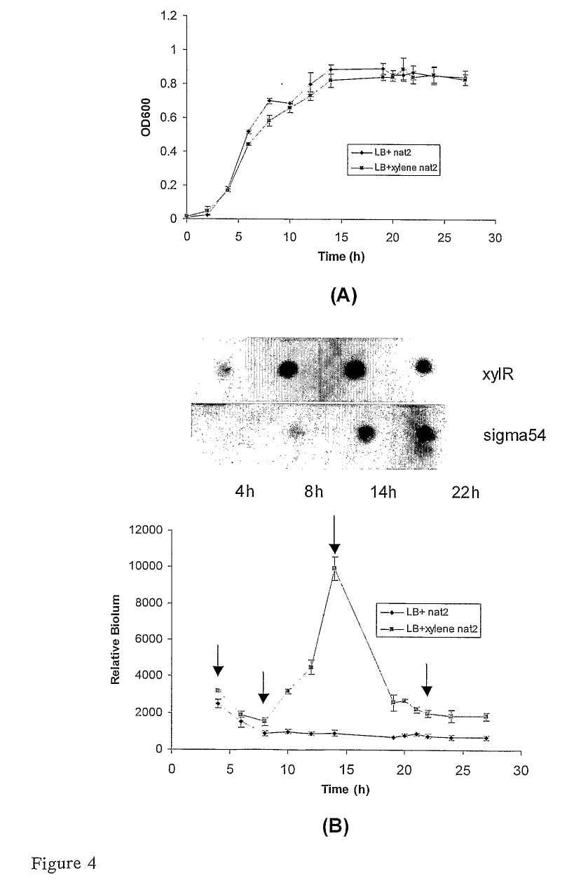

FIG. 4 illustrates mRNA transcription levels of xylR and σ⁵⁴ monitored by Northern blotting. Pu activity is associated with σ⁵⁴ transcription level. FIG. 4A depicts the growth curve of samples, and FIG. 4B shows a comparison of Northern blotting of mRNA and Pu activity.

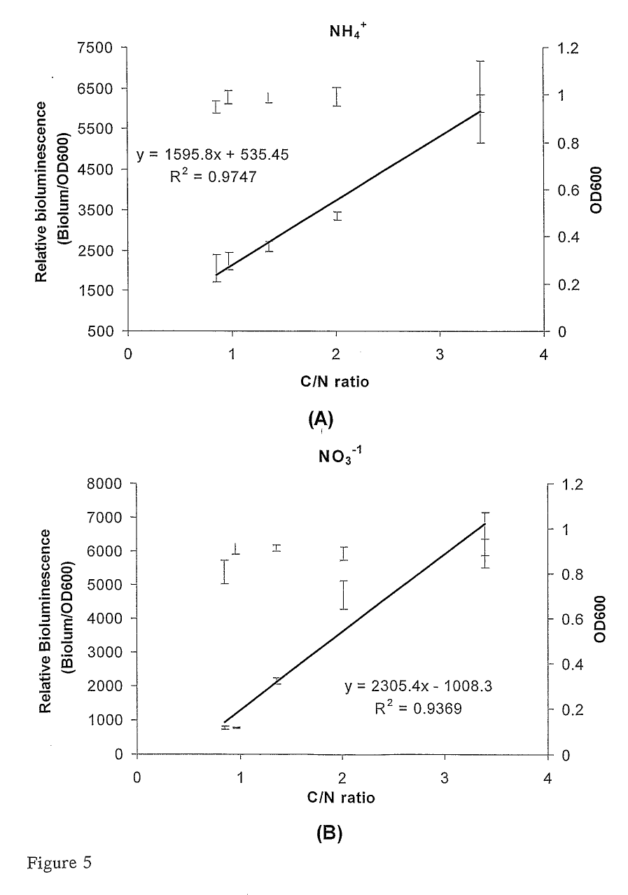

FIG. 5 illustrates the effect of carbon and nitrogen on Pu activity of Acinetobacter baylyi ADP1_lux_xylR. FIG. 5A shows the reading at OD600 and FIG. 5B shows the relative bioluminescence of five treatments.

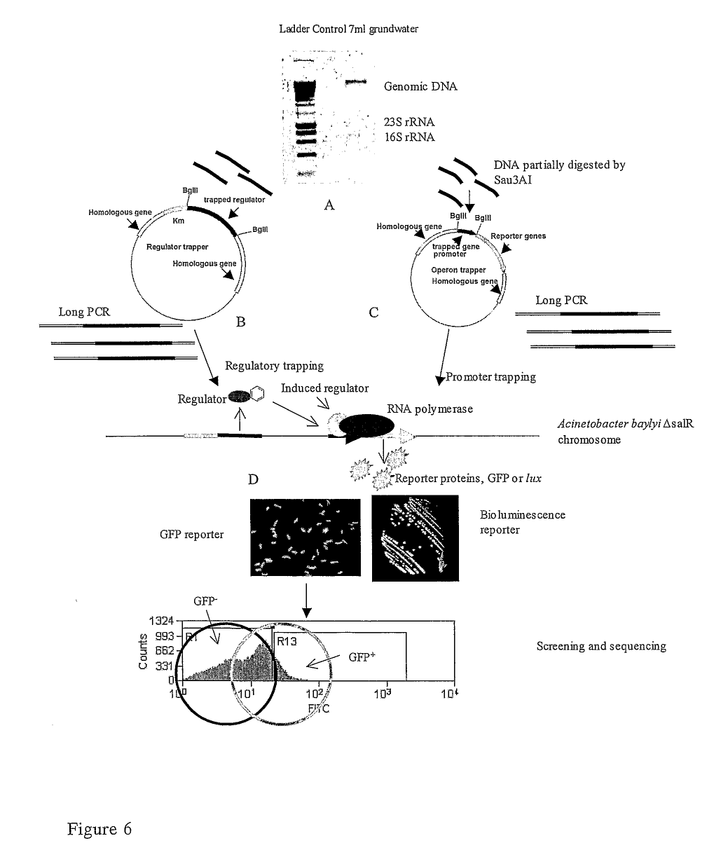

FIG. 6 illustrates schematically a method for trapping a gene encoding a regulatory protein and a promoter from DNA isolated from a groundwater sample.

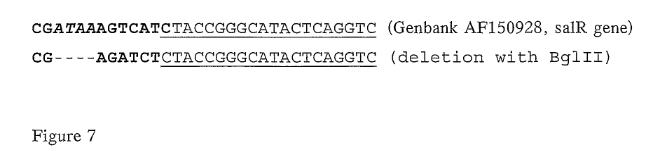

FIG. 7 shows a partial sequence of the salR gene in Acinetobacter baylyi, together with details of a mutation which removes 4 bases from the gene and reduces leaky expression of the gene.

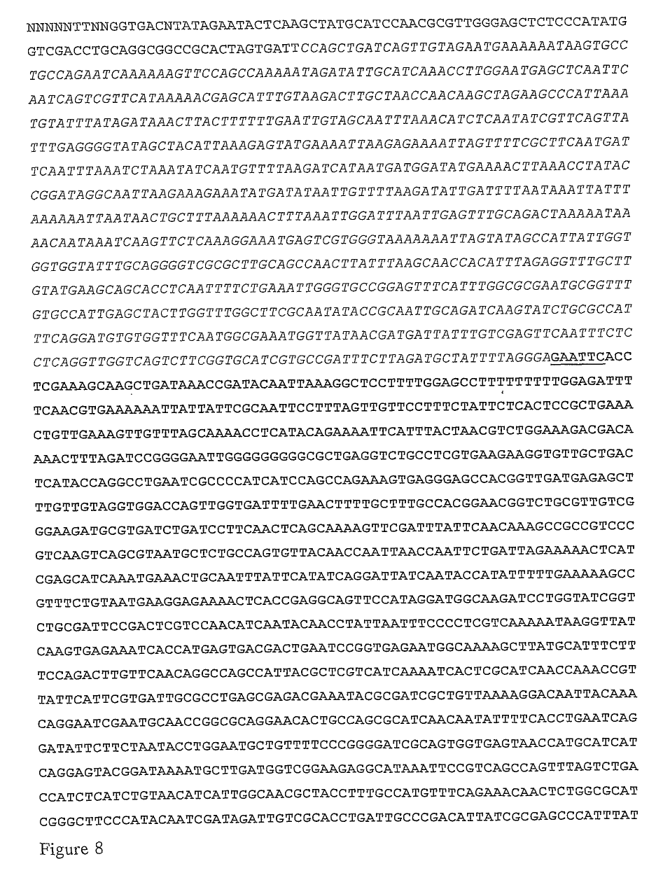

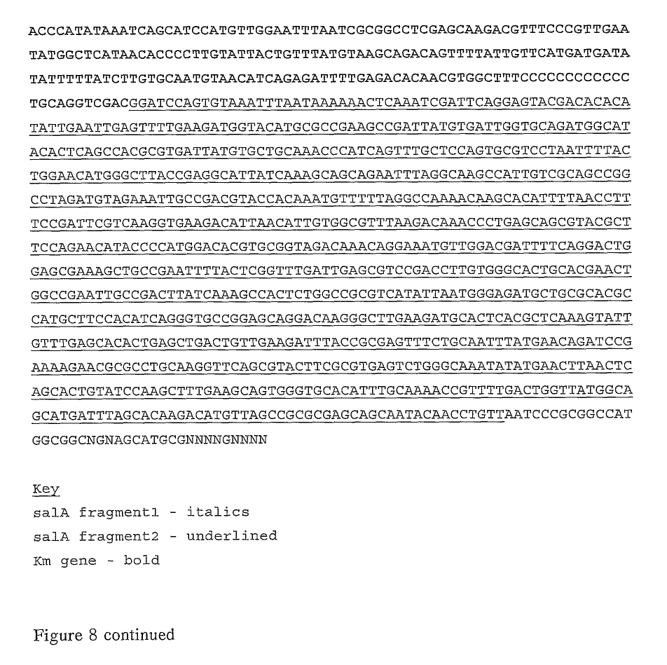

FIG. 8 is a sequence listing for salA fragments which may be used to integrate a DNA sequence into a chromosome of a host organism. The sequence includes two SalA fragments, fragments 1 and 2, flanking a kanamycin gene. This sequence can be used to. integrate and trap a potential gene encoding a regulatory protein. The sequence given reflects part of the sequence of psalA-Km in FIG. 1A.

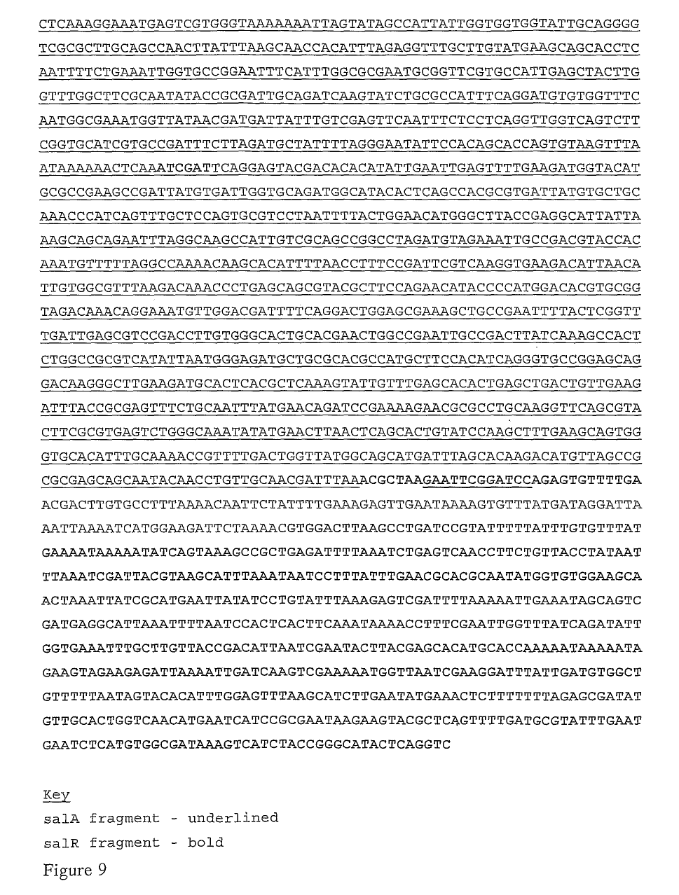

FIG. 9 is a sequence listing for salA and salR fragments which may be used to integrate a DNA sequence into a chromosome of a host organism. This sequence can be used to integrate and trap a potential promoter. The sequence given reflects the part of the sequence of pSalR-lux in FIG. 1A, before the lux genes are integrated.

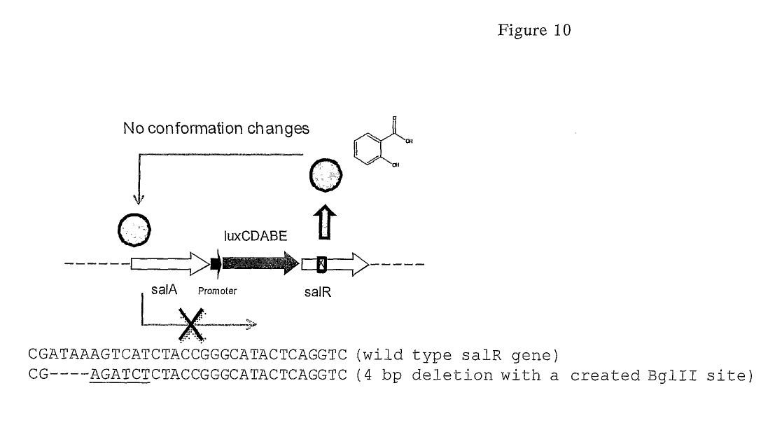

FIG. 10 is a schematic illustration of the method used to produce a mutant Acinetobacter sp strain with a mutated salR gene.

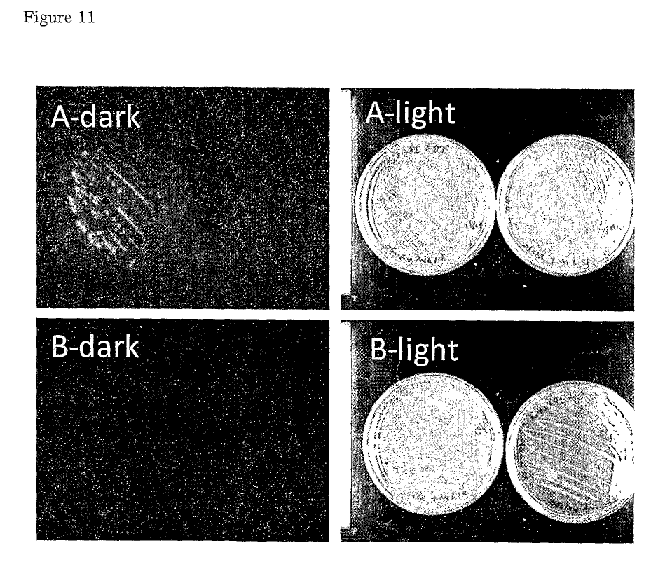

FIG. 11 depicts a positive transformant A produced by the trapping method of Example 3 in which the sal operon is activated in the presence of salicylate, and a negative transformant B produced by the method of Example 3 in which the sal operon is not activated in the presence of salicylate. Activation of the sal operon is demonstrated by bioluminescene. In the images depicted, the left-hand images are samples in the dark which illustrate that the positive transformant A is bioluminescent, this is due to the expression of the lux genes linked to the salA promoter. The right-hand images are samples in the light and illustrate that many more transformant colonies grew than were bioluminescent. The left plate contains LB+tet+salicylate, and the right-hand plate contains LB+tet.

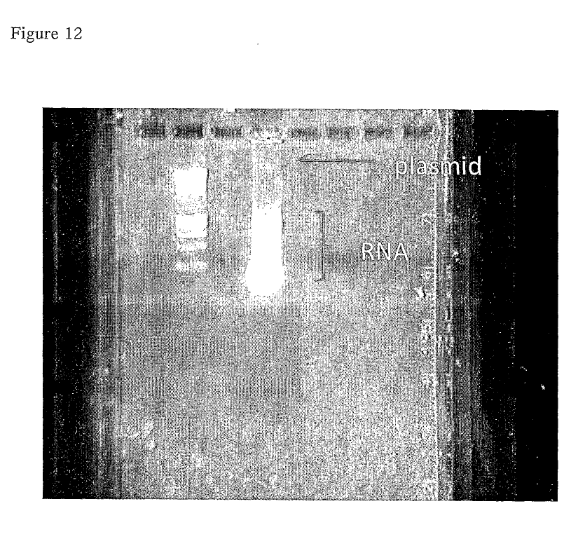

FIG. 12 is a DNA separation gel showing that the pRK415 plasmid carrying the trapped salR regulatory gene can be extracted from a positive transformant of Acinetobacter sp. ΔsalR mutant

### EXAMPLE 1

### Production of a Bacterial Biosensor Reactive to Xylene/Toluene

A bacterial biosensor reactive to xylene/toluene was produced using the method of the invention. To produce this biosensor the xylR gene and Pu promoter from a TOL plasmid, pWW0, of Pseudomonas putida were integrated into the chromosome of the bacterial host Acinetobacter baylyi ADP1. More specifically, the Acinetobacter sp. strain ADPWH_Pu_lux_xylR was constructed by fusing xylR, Pu and luxCDABE into the chromosome of A. baylyi ADP1. Activity of the Pu promoter was monitored by determining the levels of bioluminescence arising due to expression of the lux genes operably linked the Pu promoter.

The xylR gene from the TOL plasmid, pWW0, of Pseudomonas putida, encodes a toluene/xylene sensing regulation protein (XylR) and Pu is a σ⁵⁴-dependent promoter which is activated by binding of XylR (Cases et al (1996) Molecular Microbiology 19:7-17).

The Pu promoter regulation system is complex and is controlled by many factors (Cases et al (2005) Nature Reviews Microbiology 3:105-118). First, it is generally observed that the activity of the Pu promoter is dependent on the bacterial growth phase. In the exponential phase, Pu activity is repressed (exponential silencing) while it is rapidly activated on entry into stationary phase. It has been shown that the performance of σ⁵⁴ and integration host factor (IHF) are growth-phase-dependent although the level of σ⁵⁴ in some hosts, such as Pseudomonas putida is constant at different growth stages. Secondly, the presence of readily utilizable carbon sources, such as glucose induces catabolic repression, thereby inhibiting Pu activity in Pseudomonas and E. coli hosts. It has been demonstrated that the intermediate metabolites, glucose-6-phosphate dehydrogenase and/or 6-phosphogluconolactonase, act as signals for Pu repression in Pseudomonas putida (Velazquez et al (2004) Journal of Bacteriology 186:8267-8275). Other factors, such as alarmone (p)ppGpp and temperature can also affect Pu activity. However, it is not completely understood why Pu activity suddenly increases at stationary phase or which factors play the key roles in the regulation.

By cloning the xylR/Pu regulation system into another host, in this case Acinetobacter baylyi a good bacterial biosensor for xylene was rapidly produced.

Acinetobacter sp. ADP1, recently classified to Acinetobacter baylyi ADP1, is a soil bacterium with the capability to utilize a broad range of carbon sources (Young et al (2005) Annual Review of Microbiology 59). Acinetobacter and Pseudomonas belong to the same genus, A. baylyi ADP1 genome size is 3.6 Mb and has GC content is 40% in contrast to Pseudomonas which has a genome of approximately 5.4 Mb and an average GC content of 62%.

Studies on ADPWH_Pu_lux_xylR show that, like the performance in its original host of Pseudomnonas putida, the activity of the Pu promoter can be induced by toluene, o-, m-, p-xylene. These studies also demonstrate that a heterologous promoter and a heterologous gene encoding a regulatory protein can be integrated into Acinetobacter and be functional.

Material and Methods

All experiments were carried out in triplicate.

Chemicals, Bacterial Strains and Culture Media

Chemicals were all obtained from Sigma-Aldrich Co. The bacterial strains and plasmids used in this study are listed on Table 1.

TABLE 1

| Bacteria and Plasmid | Description | Reference |
|---|---|---|
| Acinetobacter sp. Strains | | |
| ADP1(BD413) | Wild type | Juni, E., and |
| | | A. Janik. 1969. |
| | | Journal of |
| | | Bacteriology |
| | | 98: 281-288) |
| ADPW67 | salA::Km^{r}, Km gene is inserted into ClaI site of | Jones, R. M., et |
| | salA | al. 2000. Journal |
| | | of Bacteriology |
| | | 182: 2018-2025 |
| ADP1_pu_lux | luxCDABE (~5.8 kb) genes are inserted | This study |
| | in EcoRI site created between salA and | |
| | salR, obtained by transformation of | |
| | ADPW67 and pSalAR lux. | |
| ADP1_pu_lux_xylR | xylR gene is inserted in EcoRI site created | This study |
| | in salA, obtained by transformation of | |
| | ADP1_pu_lux6 and pSalA_xylR_km. | |
| E. coli strains | | |
| JM109 | High efficiency competent cells | Promega |
| Plasmids | | |
| pGEM-T | Amp^{r}, T7 and SP6 promoters, lacZ, | Promega |
| | vector | |
| pRMJ2 | Source plasmid for Km gene (1654 bp) | (Jones, R. M., |
| | | and P. A. |
| | | Williams. 2003. |
| | | Applied and |
| | | Environmental |
| | | Microbiology |
| | | 69: 5627-5635 |
| pSB417 | luxCDABE source plasmid, luxCDABE | Winson, M. K., |
| | from Photorhabdus (Xenorhabdus) | S. et al. 1998. |
| | luminescens ATCC2999 (Hb strain) | Fems |
| | | Microbiology |
| | | Letters |
| | | 163: 193-202. |
| pMC2 | Source of Pu promoter | Inouye, S., A. |
| | | et al. 1983. |
| | | Journal of |
| | | Bacteriology |
| | | 155: 1192-1199 |
| pTS174 | Source of xylR and its native promoter | Inouye, S., A. |
| | | et al. 1983. |
| | | Journal of |
| | | Bacteriology |
| | | 155: 1192-1199 |
| pSalAR_lux | luxCDABE (5846 bp) genes cut out from | Huang, W. E., |
| | pSB417 by EcoRI are inserted EcoRI site | H. et al. 2005. |
| | created between salA and salR of | Environmental |
| | pSalAR_BE | Mcirobiology |
| | | 7: 1339-1348 |
| pSalAR_lux_pu | Pu gene was inserted into a EcoRI site of | This study |
| | pSalAR_lux | |
| pSalA_BE | Whole salA (1791 bp) cloned into pGEM- | This study |
| | T, a EcoRI and a BamHI restriction sites | |
| | were created by overlap extension PCR. | |
| pSalAR_Km | Km gene (1654 bp) gene cut out from | This study |
| | pRMJ2 by EcoRI and BamHI are inserted | |
| | EcoRI and BamHI sites of pSalA_BE | |
| pSalA_Km_xylR | xylR with its native promoter cut out from | This study |
| | pxylR by EcoRI and cloned into | |
| | pSalA_Km. | |

Unless otherwise stated all chemicals were obtained from Sigma-Aldrich Co. and were Analytical grade reagents. Luria-Bertani (LB) medium or minimal medium (MM) was used for the cultivation of bacteria as appropriate. Salicylate agar (SAA) medium, when required, was prepared using 2.5 mM salicylate (sodium salt) as a sole carbon source and solidified within 1.4% noble agar containing minimal medium. When appropriate, ampicillin (Amp) or kanamycin (Km) was used at final concentration of 100 and 50 μg/ml respectively for Escherichia coli. Kanamycin was added at 10 μg/ml for Acinetobacter baylyi ADP1.

Plasmid Construction

Constructing pSalAR_pu_lux (FIG. 1A, step 1)

The Pu promoter fragment (320 bp) was excised from pUC2 using EcoRI (not shown) and ligased with EcoRI partially digested pSalAR_lux (FIG. 1A, step 1). After ligation, the plasmid was transferred into E. coli competent cells JM109 by heat shock (Promega™ Co. manufacturer's manual) and then spread on LB Amp (100 μg/ml) for selection. Sixteen colonies were randomly chosen, amplified by PCR using primers salA_end_for and luxC_rev (Table 2), with an initial denaturation at 95° C. for 5 min, followed by 35 cycles of 94° C. for 1 min, 58° C. for 1 min, and 72° C. for 1 min, and a final additional 72° C. for 10 min to finish extension. After PCR amplification, 10 μl of PCR product was loaded on a 1% agarose gel. Four desirable PCR fragments (around 539 bp) were cut out from the gel, purified (Qiagen™ gel cleaning Kit) and sequenced (CEQ 2000XL, Beckman Ltd.). Based on the results of these sequences, colonies containing Pu in the same orientation as luxCDABE were selected and the plasmid carried was designated as pSalAR_pu_lux (FIG. 1A, step 1).

Constructing pSalA_km_xylR

Overlap Extension PCR (OEP) to Create Restriction Cut Sites.

EcoRI and BamHI restriction sites were created within the salA gene by overlap extension PCR as previously described (Huang, W. E. et al., (2005) Environmental Microbiol. 7, 1339-1348). Specifically, PCR amplifications were performed with an initial denaturation at 95° C. for 5 min, followed by 35 cycles of 94° C. for 1 min, 58° C. for 1 min, and 72° C. for 1 min, and a final additional 72° C. for 10 min to finish extension. Two salA fragments (salA1 and salA2) were separately amplified by colony-PCR using the primer pairs salA_flank_for-salA_BE_rev and salA_BE_fwd-salA_revH (Table 2).

TABLE 2

| Primers | Sequence (5′ → 3′) | note |
|---|---|---|
| salA_flank_for | CCAGCTGATCAGTTGTAGAATG | Outside salA |
| | | gene |
| salA_EB_for | GATGCTATTTTAGGGAGAATTC | Created EcoRI |
| | CACGGATCCAGTGTAAGT | and BamHI |
| | | sites |
| salA_EB_rev | ACTTACACTGGATCCGTGGAAT | Created EcoRI |
| | TCTCCCTAAAATAGCATC | and BamHI |
| | | sites |
| salA_revH | AACAGGTTGTATTGCTGCTCGC | The site |
| | | between salA |
| | | and salR |
| luxC_rev | GAGAGTCATTCAATATTGGCAG | Internal to |
| | G | luxC gene |
| salAR_rev | GACCTGAGTATGCCCGGTAG | |
| luxE_for | TGGTTTACCAGTAGCGGCACG | Internal to |
| | | luxE gene |
| xylR1_for | TGGATTTCAGTTAATCAATTGG | Forward |
| | T | flanking xylR |
| xylR_rev | CTATCGGCCCATTGCTTTCAC | Reverse |
| | | flanking xylR |
| Sigma54_for | ATGAAGTTATCTGTTGGATTGA | |
| | AAGTCG | |
| Sigma54_rev | CACGATATTTTGCAACGGTTCT | |
| | AC | |

PCR products were isolated from a 1% agarose gel, cleaned and purified according to the manufacturer's instructions (QIAquick™ gel extraction kit, Qiagen™ Co.). To fuse the two salA fragments, a PCR amplification (using the same reaction conditions as above, except extension time 72° C. for 2 min) was carried out which contained 1 μl of each diluted (1:100) salA1 (907 bp) and salA2 (924 bp) fragments and primers salA_flank_for and salA_revH (Table 2). The PCR product of the new salA fragment with EcoRI and BamHI restriction sites was purified according to manufacturer instructions (QIAquick™ gel extraction kit, Qiagen Co.), and then cloned into pGEM-T (Promega™ Co.), the plasmid was designated as pSalA_BE (FIG. 1A).

Construction of pSalA_Km_xylR (FIG. 1, step 3)

The pSalA_BE was digested with EcoRI and BamHI. The kanamycin gene (1472 bp) was excised from pRMJ2 (not shown) by EcoRI and BamHI and fused into pSalAR_BE to create pSalA_Km (FIG. 1A). The ligation mixture was transferred into competent cells (E. coli JM109) and transformants were obtained using selection plates of LBA with Amp (100 μg/ml) and Km (50 μg/ml).

The pSalA_Km was digested with EcoRI. The xylR gene fragment (2399 bp) was excised from pxylR (not shown) by EcoRI and fused into pSalAR_Km to create pSalA_Km_xylR (FIG. 1A, step 3). The ligation mixture was transferred into competent cells (E. coli JM109) and transformants were obtained using selection plates of LB agar (LBA) with Amp (100 μg/ml) and Km (50 μg/ml). Colony-PCR was performed to confirm that xylR had been fused into the plasmid using primer pairs xylR1_for and xylR_rev (Table 2). PCR amplifications were performed with initial denaturation at 95° C. for 5 min, followed by 35 cycles of 94° C. for 1 min, 50° C. for 1 min, and 72° C. for 2 min, and a final additional 72° C. for 10 min to finish extension.

Gene Transformation

Preparation of competent cells of Acinetobacter sp. ADP1 was performed as described in Palmen et al. 1993. Journal of General Microbiology 139: 295-305. Briefly, Acinetobacter sp. strain ADPW67 or ADP1_pu_lux served as the recipient and was grown in 5 ml LB at 30° C. overnight, with shaking at 200 rpm. Two hundred microlitres of culture was then diluted into 5 ml fresh LB medium and incubated for 2 hours to make the cells competent. For transformation, 2 μg of plasmid of pSalAR_pu_lux or SalA_Km_xylR was added to 0.5 ml competent cells (10⁹ cells/ml) and incubated for 2 hours. Subsequently, the cultures were plated on appropriate media for selection of transformants.

Creating Acinetobacter sp. ADP1_pu_lux (FIG. 1A, step 2)

The protocol for integration of the Pu promoter is shown in FIG. 1A. Acinetobacter sp. strain ADPW67 has a kanamycin gene inserted into the salA gene and it cannot grow on SAA plates which provide salicylate as a sole carbon source. After integration, the salA gene which is disrupted by the Km gene, was replaced by pSalAR_pu_lux providing a functional salA gene and enabling the transfromants to grow on SAA.

To confirm Pu promoter and luxCDABE integration into the chromosome of Acinetobacter sp. ADP1, 16 colonies were randomly chosen from the SAA plate and PCR reactions were performed using a chromosomal flanking primer and an internal primer. Specifically, salA_flank_for/luxC₁₃ rev primer pairs are for Pu and salAR_rev/luxE_fwd (table 2) primer pairs for luxCDABE.

Transformants were tested by colony-PCR followed by gel cleaning and sequencing and designated Acinetobacter sp. strain ADP1_pu_lux

Creating Acinetobacter sp. ADP1_pu_lux_xylR (FIG. 1A, step 4)

To integrate xylR and the Km gene into the chromosome of Acinetobacter ADP1_pu_lux, plasmid pSalA_Km_xylR was mixed with competent cells of Acinetobacter ADP1_pu_lux as mentioned above. The mixture was incubated at 30° C. for 2 hrs and spread on LB with 10 μg/ml kanamycin agar plate for selection. PCR was carried out to confirm integration of xylR into ADP1_pu_lux using the primer pair xylR1_for and xylR_rev (Table 2).

Nucleotide Sequencing and Sequence Analysis

All DNA samples (PCR products or plasmids) were sequenced using dye terminator sequencing on an Applied Biosystems 3730 DNA analyzer according to the manufacturers instructions. DNA sequence analysis was carried out using Blastn for confirmation of sequence homology. Subsequently, the DNA sequences were aligned and edited using BioEdit™ (Tom Hall, Department of Microbiology, North Carolina State University) to confirm correct insertions. The plasmid pSalAR_Km_xylR has been fully sequenced and submitted to the National Center for Biotechnology Information (NCBI) and the accession number is DQ202262.

Methods to Monitor and Study Acinetobacter sp. ADP1_Pu_lux_xylR Induction

Monitoring Bacterial Growth and Bioluminescence

Pu promoter activity was monitored by measuring relative bioluminescence (bioluminescence divided by OD600). For each measurement, at each time point 100 μl samples were analyzed in triplicate in a well of a black, clear-bottom 96-well microplate (Fisher Scientific). At OD600 and Bioluminescence were measured using a Synergy HT Multi-Detection Microplate Reader (Bio-Tek™).

Different Inducers and Temperatures

A single colony of Acinetobacter sp. ADP_pu_lux_xylR was separately inoculated into 5 ml LB medium in a 30-ml glass universal tube. Toluene, o-, m-, p-xylenes, phenol, benzene, naphthalene, 2-, 3-, or 4-hydroxybenzoic, benzoate or catechol was added into LB (100 μM) to assay induction of Pu. Samples were incubated at 20, 28, 30, 34 or 37° C. with shaking at 150 rpm. Samples were repeatedly loaded on black 96-well microplate for bioluminescence and OD600 measurement for 30 hours of incubation.

Growing in Minimal Medium with Glucose or Succinate as Sole Carbon Source

To examine the effect of catabolic repression Acinetobacter sp. ADP1_pu_lux_xylR was inoculated into 5 ml MM supplemented with 50 mM glucose or succinate as a sole carbon source in a 30-ml glass universal tube. Toluene, o-, m-, p-xylenes were added separately into the media (500 μM) as inducers. The bacterial samples were incubated at 28° C. shaker with shaking at 150 rpm. To identify the signal molecules for Pu repression 6-phosphogluconic acid trisodium salt (Sigma-Aldrich Co.), which was supposed to be a signal molecule for repression, was added into MM-glucose medium at a final concentration of 0.1, 1, 10, 30, 60 or 300 μM, after bioluminescence from Acinetobacter sp. ADP1_pu_lux_xylR had been maximally induced by m-xylene. For each treatment, three replicates were carried out. Samples were taken and measured for bioluminescence and OD600 every 30 min.

The Effect of Carbon-Nitrogen Ratio on Pu Activity

To examine the effect of carbon and nitrogen on Pu activity, Acinetobacter sp. ADP1_pu_lux_xylR was separately inoculated into five different media: (1) 1:2 diluted LB medium only; (2) 1:2 diluted LB medium with 500 μM m-xylene; (3) 1:2 diluted LB medium with 500 μM m-xylene and 19 mM NH4Cl; (4) 1:2 diluted LB medium with 500 μM m-xylene and 20 mM glucose; and (5) 1:2 diluted LB medium with 500 μM mxylene, 20 mM glucose and 19 mM NH₄Cl. The bacterial samples were incubated at 28° C. with shaking at 150 rpm. Samples were repeatedly loaded on black 96-well microplate for bioluminescence and OD600 measurement for 25 hours of incubation.

Northern Dot Blotting

Northern dot blotting was used to examine xylR and σ⁵⁴ RNA transcription levels in Acinetobacter sp. ADP1_Pu_lux_xylR during induction. Acinetobacter sp. ADP1_Pu_lux_xylR was incubated in LB at 28° C. with shaking at 150 rpm. m-xylene (500 μM) was added into LB medium and sampled at 4, 8, 14 and 22 hrs. Three replicates were carried out. At each time point, a 1 ml aliquot was removed from which total cellular RNA was extracted according to the manufacturer's instruction (Qiagen™ RNA/DNA mini kit). PCR products of xylR and σ⁵⁴ were used as DNA templates for primer pairs xylR1_for-xylR_rev and sigma54_for-sigma54_rev (Table 2). PCR products of xylR and σ⁵⁴ were used as the templates and labelled separately (Ambion Co.). Briefly, 1 μl of 10 mM EDTA was added into a PCR tube with 9 μl DNA product (100 ng/μl). The PCR tubes were placed in a boiling water bath for 10 min and quickly cooled by dry ice to generate single strand DNA probes. Subsequently, 1 μl Psoralen-Biotin was added into each tube immediately and mixed thoroughly, and then transferred into a 96 well plates which was rapidly transferred to the dark. The probes were labelled under 365 nm UV light for 45 min. Finally the probes were purified using the protocol provided by the kit (Ambion™'s nonisotopic labeling kit) and stored in a −80° C. freezer. BrightStar-Plus™ positively charged nylon membrane (Ambion Co.) was used for dot blotting. The same amount of total RNA of each sample was loaded onto the membrane and fixed at 80° C. for 15 mins. Hybridization was performed by adding 30 ng/ml denatured probes into the NorthernMax™ Hybridization (Ambion) buffer and incubated at 42° C. for 12-20 hrs after the membrane had been prehybridized for 30 min. The hybridized membrane was washed by 2×SSC and 0.5% SDS twice for 30 min and then detected according to the manufacture's instructions (BrightStar BioDetect™ Ambion Co.). The membrane was put into a cassette loaded with Kodak™ scientific imaging film (Kodak Co.) in the dark. The film was exposed for one hour, manually washed, and dried at room temperature.

Results and Discussion

Using the aforementioned methods and material a toluene/xylene biosensor was generated by inserting xylR and pu-luxCDABE into the chromosome of Acinetobacter baylyi ADP.

The recipient Acinetobacter baylyi ADP1 cells took up the naked foreign DNA and integrated it into the chromosome by homologous recombination. The salA promoter on the Acinetobacter baylyi ADP1 chromosome is capable of transcribing large inserts (>5.8 kb) located between salA and salR. The expression of salA and salR was shown not to be affected by the presence of an insert. Mutants carrying inserts were able to grow on a SAA plate where salicylate was used as a sole carbon source (Huang et al (2005) Environmental Microbiology 7:1339-1348). In this example, the Pu promoter was fused to pSaIAR _lux to generate the plasmid pSalAR_pu_lux (FIG. 1A, step 1). pSalAR_pu_lux has homologous fragments of salA and salR flanking Pu-luxCDABE, which served as the DNA donor. The recipient, Acinetobacter mutant ADPW67, which contains a disrupted salA with a Kanamycin gene insertion, cannot grow on a SAA plate. After transformation, the disrupted salA gene of ADPW67 was recovered by homologous recombination with pSalAR_Pu_lux and transformants acquired the capability to grow on SAA. Simultaneously, Pu-luxCDABE was introduced between the salA and salR of Acinetobacter ADP1_Pu_lux (FIG. 1A, step 2).

To introduce xylR into the chromosome, plasmid pSalA_Km_xylR (FIG. 1A, step 3) was constructed and xylR-km was flanked with two homologous fragments of salA. Gene transformation was carried out, using pSalA_Km_xylR as a DNA donor and Acinetobacter ADP1_Pu_lux as a recipient. The transformants were screened by growing on LB with 10 μg/ml Km and designated as Acinetobacter sp. ADP1_Pu_lux_xylR (FIG. 1A, step 4). A terminator from the Km insert was included between the xylR and Km genes preventing RNA polymerase from reading through to luxCDABE without activation of the Pu promoter. The chromosomal structure of the inserts of Acinetobacter was confirmed by PCR and sequencing.

Acinetobacter sp. ADP1_pu_lux_xylR Response to Toluene/Xylenes

The xylR/Pu regulation system from the TOL plasmid of P. putida works in the heterologous host A. baylyi ADP1. FIG. 1B shows that Acinetobacter ADP1_Pu_lux_xylR expressed strong bioluminescence when it was induced by xylene vapors while uninduced cells remained dark indicating the successful transformation and operation of Pu-xylR regulation system in the host of Acinetobacter strain ADP1_Pu_lux_xylR. In other words, Acinetobacter ADP1_Pu_lux_xylR can be regarded as a biosensor for toluene and xylenes.

A large number of regulation systems have been found that mediate catabolic pathways, and their genetic organizations often include a promoter and a regulatory gene (Cases and Lorenzo (2005) Nature Reviews Microbiology 3:105-118; Tropel and van der Meer (2004) Microbiology and Molecular Biology Reviews 68:474-). A. baylyi ADP1 can express heterologous genes without difficulties.

The skilled man will appreciate that the approach described with reference to xylene/toluene could be used with other regulatory operons to produce other bacterial biosensors by replacing the promoter and regulation gene. In this way, this system can be applied to construct a wide range of inducible biosensors.

Like its original host of P. putida, Pu activity within strain ADP1_pu_lux_xylR was only activated in the stationary phase regardless of the presence of inducers, demonstrating a pattern of exponential silencing (FIG. 2) (Cases and de Lorenzo. (2001) EMBO Journal 20:1-11 and (2005) Nature Reviews Microbiology 3:105-118). However, this contrasts with a previously reported Escherichia coli system in which the Pu promoter was induced shortly after cells were exposed to xylene without experiencing exponential silencing (Willardson et al (1998) Applied and Environmental Microbiology 64:1006-1012). Unlike tod systems (Applegate et al (1998) Applied and Environmental Microbiology 64:2730-2735), the Pu promoter in Acinetobacter ADP1_pu_lux_xylR cannot be induced by phenol, benzene, naphthalene, 2-hydroxybenzoic (salicylic acid), 3-hyroxybenzoic, 4-hydroxybenzoic, benzoate or catechol following 30 hrs incubation. This suggests that the Pu-xylR system specifically responds to toluene, xylenes or their analogs. The methyl group of the benzene ring could be important to induce the correct conformation of the xylR protein.

Catabolic Repression of Acinetobacter sp. ADP1_pu_lux_xylR

In their natural habitats, when multiple carbon sources are available, bacteria prefer to utilize favourable substrates to economize cellular metabolism. Bacteria achieve this regulation through carbon catabolite repression (CCR). In the presence of certain (favourable) carbon sources (typically glucose), bacteria will repress the machinery needed for the assimilation of other less labile carbon sources (Bruckner and Titgemeyer (2002) Fems Microbiology Letters 209:141-148.). Previous reports have demonstrated that glucose restrains Pu activity in Pseudomonas sp. and E. coli. (Cases et al (1999) Journal of Biological Chemistry 274:15562-15568). In this study, Acinetobacter sp. ADP1_Pu_lux_xylR was inoculated into minimal medium (MM) with 50 mM glucose or succinate as sole carbon sources and toluene, m-, p- or o xylene as inducers. In this system, glucose did not repress Pu activity, whereas succniate did (FIG. 3A). Case et al. showed that disrupting pstN, encoding the IIANtr protein (of the phosphotransferase system (PTS)), made Pseudomonas putida lose the ability to regulate glucose repression. In contrast to Pseudomonas sp. and E. coli, A. baylyi ADP1 does not contain a glucose transport phosphotransferase system. Glucose cannot enter into the cytoplasm of A. baylyi ADP1 and is only oxidized in the periplasm. This characteristic of glucose assimilation might contribute to the lack of response to catabolite repression by glucose in Acinetobacter ADP1 (FIG. 3A). This suggests that glucose might be directly involved in catabolite repression.

Acinetobacter ADP1 does not encode genes for a glucose-6-phosphate dehydrogenase nor a 6-phosphogluconolactonase. Velazquez et al. suggests that 6-phosphogluconate and/or 2-dehydro-3-deoxyphosphogluconate might be the signal molecules for Pu repression in Pseudomonas putida. In this study, deliberately adding sodium salt 6-phosphogluconate to strain ADP1_Pu_lux_xylR did not affect Pu activity (data not shown). This observation suggests that 6-phosphogluconate alone is not sufficient to repress Pu activity and it may help narrow down candidates of signal molecules for Pu repression. Because of the advantage of easy gene manipulation, A. baylyi ADP1 would enable signal molecules to be identified which mediate Pu promoter regulation through knocking out genes and inserting new functional genes.

The optimal temperature for Acinetobacter sp. ADP1 and the xylR/Pu original host Pseudonzonas putida, is 30° C. In this study, it was found that the best temperature for Pu promoter activity was 28° C. which was 2-5 times higher than at 20, 34 or 37° C. (FIG. 3B).

Transcription Regulation of Pu/xylR in the Acinetobacter System.

Both xylR and σ⁵⁴ mRNA levels in Acinetobacter strain ADP1_Pu_lux_xylR were examined by Northern blotting at different time points (FIG. 4). Although m-xylene was introduced to the LB medium at the outset, Pu activity remained silenced until cells entered stationary phase (FIG. 4). As with previous observations (Jurado et al (2003) Journal of Bacteriology 185:3379-3383: Ramos et al (1997) Annual Review of Microbiology 51:341-373), Northern blotting confirmed that mRNA transcription levels of both xylR and in ADP1_Pu_lux_xylR were constitutively transcribed during the growth phase (FIG. 4B).

However, the mRNA level of σ⁵⁴ varied at different times. At 8-hr, the growth curve (FIG. 4A) shows that cells have started to enter stationary phase, at which time point the mRNA level of xylR has reached the same level as the 14 or 22-hr point (FIG. 4B). In contrast, the σ⁵⁴ transcription level and Pu activity were relatively low in comparison with the 14 or 22 hr time points (FIG. 4B). At 14-hr, σ¹⁴ mRNA levels increase, and Pu activity reached its highest level (FIG. 4B). This indicates a direct link between expression, Pu activity and growth phase as Cases et al. found in a Pseudomonas putida host (Cases et al (1996) Molecular Microbiology 19:7-17.).

### EXAMPLE 2

### Method to Trap a Gene Encoding a Regulatory Protein and a Promoter from a Water Sample to Produce a Biosensor for a Specific Compound

FIG. 6 shows schematically a method to trap a gene encoding a regulatory protein and a promoter from a water sample to produce a biosensor for a specific compound

First of all, the total DNA from a 7 ml sample of groundwater would be extracted to give greater than 10 kb of DNA.

The protocol used to extract nucleic acid from a groundwater sample is as follows, and can be scaled to suit the amount of ground water used. Initially 50 ml groundwater is passed through 0.22 μm filter. The filter is then placed into a BIO-101™ tube (Q-biogene™). 1 ml of DNA extraction buffer is then added to the tube and incubated in a 65° C. water bath for 30 min. The DNA extraction buffer contains 100 mM Tris_HCl [pH8.0], 100 mM sodium EDTA [pH8.0], 100 mM phosphate buffer [pH8.0], 1.5M NaCl, 1% CTAB, and water. The tubes are then placed in a Fast-prept™ beadbeater for 30 seconds at speed 5.5 to lyse the cells. The tubes are then cooled on ice and centrifuged at 14,000 rpm for 5 min (chilled). The water phase is extracted and an equal volume of chloroform:isoamyl alcohol(24:1) is added and mixed well. The tubes are centrifuged for 5 minutes at 14,000 rpm. The top layer is extracted and DNA is precipitated by adding 0.6 volume of isopropanol (or 30% PEG 6000/1.6M NaCl) and mixing well. The tubes are left to stand on the bench for 1-2 hours at room temperature, and then centrifuged at 14,000 rpm for 10 min. The supernatant is poured off and the pellet is washed with 200 μl 70% ethanol. The tube is then centrifuged again at 14,000 rpm for 10 min, the ethanol is poured off and the pellet is left to dry in a vacuum machine or by air. The pellet is resuspended in 50 μl TE Buffer or water.

DNA isolated from the water is then partially digested using Sau3AI and ligated into suitable trapping plasmids. Two trapping plasmids are used. The first plasmid, termed the operon trapper (OT) plasmid, contains a reporter gene and is arranged to trap the promoter. The second plasmid, termed the regulator trapper (RT) plasmid is arranged to trap the gene encoding the regulatory protein. Both plasmids contain homologous DNA fragments of Acinetobacter baylyi ADP1.

The host organism is preferably Acinetobacter baylyi ADP1, preferably the host strain is a ΔsalR mutant. In a ΔsalR mutant the salR gene of the Acinetobacter mutant is partially deleted, which makes it incapable of growth on a minimal agar (MM) plates containing salicylate as a sole carbon source (Huang et al., (2005) Anal Chem 76, 4452-4458).

The Sau3Al digested DNA is cloned into the OT or RT plasmids, which are then amplified by transferring the plasmids to E. coli., or more simply by using long PCR (FIGS. 6B and 6C). To integrate the cloned DNA into the host organism, the plasmids or PCR products are mixed with competent cells of Acinetobacter strain ΔsalR. Integration requires two steps: (1) potential promoter integration which is selected by the restoration of salicylate growth; and (2) potential regulatory gene integration which is selected by antibiotic resistance e.g. kanamycin (FIG. 6D).

Once integrated into the chromosome of Acinetobacter, the fragment of DNA which could be the gene encoding the regulatory protein (that is the DNA from the RT plasmid) is constitutively expressed. The cloned DNA in the OT plasmid is now integrated in the Acinetobacter chromosome operably linked to a reporter gene.

Acinetobacter with both DNA sequences integrated are then subjected to exposure to the target chemical compound. If a cell contains a regulatory protein and a promoter activated in the presence of the regulatory protein and the target chemical compound, the reporter gene will be expressed. Cells may be screened for reporter gene expression by FACS (Fluorescence Activated Cell Sorting) for GFP, or by a colony-picker for lux bioluminescence, or by an X-Gal assay for lacZ. Any cells expressing the reporter gene can then be selected and analysed. Using this method a gene encoding a regulatory protein and the corresponding promoter can be trapped, and a biosensor produced.

An Acinetobacter ΔsalR mutant, in which part of the salR gene of the Acinetobacter has been deleted making it incapable of growth on a minimal agar (MM) plates containing salicylate as the sole carbon source (Huang et al., 2005. Anal Chem 76, 4452-4458), can be made using the following method. Overlap extension PCR (OEPCR) is used to create ΔsalR fusions with required restriction sites (a suitable method is described in Huang et al., (2005) Anal Chem 76, 4452-4458). The ΔsalR fusion is then be cloned into a pGEM-T vector (Promega™) to create pΔsalR. pΔsalR is then digested with BamHI. A Km-SacB cassette cut from pRMJ1 with BamHI (Jones and Williams, Applied and Environmental Microbiology 69 (9): 5627-5635 September 2003) is then inserted into pΔsalR to create pΔsalR_SacB_Km. pΔsalR_SacB_Km is then transferred to Acinetobacter ADPWH_lux as described in Huang et al. (2005) Anal Chem 76, 4452-4458, and mutants selected using LB containing 10 μg/ml kanamycin. The selected mutants are designated as Acinetobacter ΔsalR_SacB_Km. pΔsalR is then transferred to Acinetobacter ΔsalR_SacB_Km and mutants selected using LB with 50 g/L sucrose, the selected mutants are designated as Acinetobacter ΔsalR.

### EXAMPLE 3

### Production of a Bacterial Biosensor Reactive to Salicylate from a Pool of DNA Isolated from a Naphthalene Contaminated Site

In this example a plasmid was used to trap a gene encoding the salicylate regulatory gene from a pool of DNA extracted from a sample taken from a naphthalene contaminated site. This gene may then be cloned into the chromosome of a mutant strain of Acinetobacter sp. in which the salA promoter is opereably linked to a reporter to produce a biosensor for salicylate or naphthalene. This example demonstrates that a regulatory gene can be isolated from a pool of DNA recovered from an environmental sample.

Construction of Acinetobacter sp. ΔsalR Mutant

The first step in the isolation of the salicylate regulatory gene from an environmental sample was to produce a mutant strain of Acinetobacter sp. ADP1 which did not express a functional salicylate regulatory gene. This was achieved using the following steps:
1. overlap extension PCR (OEPCR) was used to create ΔsalR fusions with required restriction sites, as previously described (Huang et al., 2005 Environmental Microbiology 7: 1339-1348);
2. the ΔsalR gene was then cloned into a pGEM-T vector to create a plasmid pΔsalR;
3. the plasmid pΔsalR was then digested with BamHI;
4. the Km-SacB cassette was isolated from pRMJ1 using BamHI (Jones and Williams, 2003 Applied and Environmental Microbiology 69: 5627-5635) and cloned into the pΔsalR plasmid to create pΔsalR_SacB_Km;
5. plasmid pΔsalR_SacB_Km was then transferred into Acinetobacter ADPWH_lux as previously described (Huang et al., 2005 Environmental Microbiology 7: 1339-1348), and mutants were selected with LB containing 10 μg/ml Km. The mutant was designated as Acinetobacter ΔsalR_SacB_Km. In Acinetobacter ADPWH_lux and Acinetobacter ΔsalR_SacB_Km the salA gene is operably linked to the reported genes luxCDABE;
6. pΔsalR was then transferred to Acinetobacter ΔsalR_SacB_Km and positive transformants were selected on LB with 50 g/L sucrose, the mutants produced were designated as Acinetobacter ΔsalR. The resultant strain is mutant for the salR gene but carries the salA promoter operably linked to the lux reporter genes. This strain can be used to screen for genes encoding a salicylate regulatory protein.

Essentially, the mutation made to the salR gene in Step 1 involves the deletion of 4 bases in the salR gene and the introduction of a BglII restriction enzyme site as indicated below:

CGATAAAGTCATCTACCGGGCATACTCAGGTC (wild type salR gene) CG----AGATCTCTACCGGGCATACTCAGGTC (ΔsalR with 4 bp deletion and a BglII site)

The deletion of the 4 bases in the salR gene results in a disrupted salicylate regulatory protein which cannot respond to salicylate, thus the salA promoter in the Acinetobacter sp. is not activated and there is no expression of the lux genes in the presence of salicylate. The effect of the inactivation of the salR gene is illustrated in FIG. 10.

However, if an active salR gene was present in the mutant Acinetobacter sp. then in the presence of salicylate, the salR regulatory protein could activate the salA promoter and induce the expression of a reporter gene operably linked to the salA promoter.

In this example the reporter is the bioluminescence genes luxCDABE. By using bioluminescence in the biosensor positive clones can be identified quickly in-situ—typically in less than 15 mins. GFP is an alternative reported but it needs more time to mature before it can be detected.

Create a Clone Library of Environmental DNA

The second stage in the isolation of the salicylate regulatory was to obtain a pool of DNA from an environmental sample. In this case the pool of DNA was extracted from a naphthalene contaminated groundwater site in the UK. The DNA may be extracted from the water using the method described in Example 2.

The isolated DNA was partially digested with Sau3AI, and cloned into pRK415—the “trapping plasmid”. pRK415 was then transformed into the to Acinetobacter sp. ΔsalR mutant by electroporation. pRK415 is able to replicate in Acinetobacter sp. ADP1 and to express any genes cloned into it. Acinetobacter sp. ΔsalR transformants were screened by plating on LB supplemented with 6 μg/ml tetracycline and 2 mM salicylate.

In an alternative embodiment, the cloned DNA may be integrated at this stage into the host chromosome, this may be achieved by including flanking sequences which are homologous to the host chromosome.

Screen the Transformants for “Trapped” Salicylate Regulation Gene

Acinetobacter sp. ΔsalR transformants growing on LB supplemented with 6 μg/ml tetracycline and 2 mM salicylate were then screened based on bioluminescence expression. The theory being that transformants able to show bioluminescence in the presence of salicylate, that is, able to activate the salA promoter operably linked to the lux genes in Acinetobacter sp. ΔsalR, must be expressing a regulatory protein of salA.

In this example the regulatory protein is expressed from a plasmid. Three bioluminescence positive transformants were found among more than 4000 transformants produced. FIG. 11 depicts bioluminescence expression from a positive transformant (Transformant A) and the lack of bioluminescence from a negative transformant (Transformant B). Transformant A demonstrates that in the presence of salicylate bioluminescence occurs, indicating that Transformant A has trapped a salicylate regulatory gene from the pool of DNA extracted from the environmental sample which is ale to compensate for the mutation in the salR gene carried by Acinetobacter sp. ΔsalR. Transformant B (which was one of 4000 negative transformants) is not activated by salicylate, as evidenced by the absence of any bioluminescence, indicating that Transformant B does not carry a trapped the salicylate regulatory gene.

Plasmid Extraction and DNA Sequencing

To confirm that Transformant A, and indeed the other two positive transformants, had trapped the salicylate regulatory gene, the pRK415 trapping plasmids from the three bioluminescence positive transformants were extracted using boiling lysis (Sambrook et al., 1989 Molecular cloning: a laboratory manual: Cold Spring Harbor Laboratory, Cold Spring Harbor, N.Y.). A sample of the extracted plasmid DNA run on an agarose gel is shown in FIG. 12. Sequencing of the extracted plasmids may be used to confirm that the cloned gene is indeed the salicylate regulatory gene.

In conclusion, this experiment demonstrates that a mutant strain of Acinetobacter sp. ADP1 can be successfully used to trap genes from an environmental sample. In this example the regulatory gene activated by salicylate was trapped/cloned using a plasmid based system, but the same principle may be used to clone other regulatory genes, and/or to clone promoters of interest.

Once trapped the regulatory gene and/or promoter of interest may be integrated into the chromosomal DNA of Acinetobacter sp. to produce a biosensor. In this example, the trapped regulatory gene may be integrated into the chromosome of Acinetobacter sp. ΔsalR (as previously described) to replace the mutated salR gene, and preferably is arranged such that it is constitutively expressed to produce a biosensor for using in detecting salicylate.

1. A method of producing a biosensor for a specific compound comprising:
(a) identifying the specific compound;
(b) obtaining a pool of DNA;
(c) at least one of:
(i) cloning fragments of DNA from the pool of DNA into a first and a second site in one or two plasmids; and integrating the cloned DNA into a chromosome of a host organism, wherein the DNA from the first site in the plasmid is integrated into the chromosome at a first position such that it will be expressed in the host organism, and DNA from the second site in the plasmid is integrated into the chromosome at a second position such that the cloned DNA is operably linked to a reporter gene; or
(ii) cloning a gene which encodes a regulatory protein responsive to the specific compound into a first position in a first plasmid; cloning a promoter, which is activated in the presence of both the regulatory protein and the specific compound, into a second position in the first plasmid or into a second plasmid; and integrating the cloned gene which encodes the regulatory protein, and the cloned promoter, into a chromosome of a host organism, wherein the promoter is operably linked to a reporter gene;
(d) applying the specific compound to the host organism; and
(e) screening for expression of the reporter gene.

2. A method of identifying both (i) a gene encoding a regulatory protein responsive to a specific compound and (ii) a promoter activated by the regulatory protein and the specific compound:
(a) identifying the specific compound;
(b) obtaining a pool of DNA;
(c) cloning fragments of DNA from the pool of DNA into a first and a second site in one or two plasmids such that DNA at the first site will be expressed when the plasmid is transformed into a host organism, and DNA at the second site is operably linked to a reporter gene;
(d) transforming a host organism with the one or two plasmids;
(e) applying the specific compound to the transformed host organism; and
(f) screening for expression of the reporter gene.

3. A method of identifying a gene encoding a regulatory protein responsive to a specific compound comprising:
(a) identifying a specific compound;
(b) obtaining a pool of DNA;
(c) cloning fragments of DNA from the pool of DNA into a plasmid;
(d) at least one of
(i) integrating the cloned DNA into the chromosome of a host organism such that the cloned DNA is expressed in the host organism, wherein the chromosome already carries a promoter operably linked to a reporter gene, and wherein the promoter is known to be activated in the presence of the specific compound and an unknown regulatory protein; or
(ii) transforming a host organism with the plasmid containing the cloned DNA, wherein the host organism carries a promoter operably linked to a reporter gene, and wherein the promoter is known to be activated in the presence of the specific compound and unknown regulatory protein;
(e) applying the specific compound to the host organism; and
(f) screening for expression of the reporter gene.

4. (canceled)

5. A method of identifying a promoter activated by a regulatory protein which is responsive to a specific compound comprising:
(a) identifying a specific compound;
(b) obtaining a pool of DNA;
(c) cloning fragments of DNA from the pool of DNA into a plasmid;
(d) at least one of
(i) integrating the cloned DNA into the chromosome of a host organism such that the cloned DNA is operably linked to a reporter gene, wherein the chromosome already carries a gene encoding a regulatory gene which is responsive to the specific compound; or
(ii) transforming a host organism with the plasmid containing the cloned DNA operably linked to a reporter gene wherein the host organism carries a gene encoding a regulatory protein which is responsive to the specific compound;
(e) applying the specific compound to the host organism; and
(f) screening for expression of the reporter gene.

6-8. (canceled)

9. The method according to claim 1, wherein the specific compound is an environmental contaminant or pollutant.

10. The method according to claim 1, wherein the pool of DNA is obtained by isolating DNA from a sample contaminated with the specific compound.

11. The method according to claim 1, wherein the pool of DNA is obtained from a sample of soil, ground water, any body of water, the air, a human or non-human body or body fluid, or any other suitable sample.

12. The method according to claim 1 wherein the cloned DNA is integrated into a chromosome of the host organism, wherein at least one of the DNA is integrated into the chromosome directly from a plasmid(s), it is amplified by PCR and the PCR fragment is integrated into the chromosome, or the cloned sequences are integrated into a chromosome in the host organism by homologous recombination.

13. (canceled)

14. The method according to claim 1, wherein at least one of the DNA integrated at the first position in the host chromosome or the second site in the plasmid is arranged to be constitutively expressed in the host organism or wherein the DNA integrated at the second position in the host chromosome or the second site in the plasmid is arranged to be located operably linked to a reporter gene.

15. (canceled)

16. The method according to claim 3, wherein the cloned DNA is constitutively expressed in the host organism.

17. The method according to claim 5, wherein the gene encoding the regulatory protein is constitutively expressed in the host organism, at least under test conditions.

18. The method according to claim 1, wherein the gene encoding the regulatory protein, and/or the promoter, are heterologous to the host organism.

19. The method according to claim 9, wherein the compound is selected from the group comprising aromatic solvents, chlorinated compounds, nitrates, and pesticides from agricultural runoff, components of fuels, solvents, propellants, pesticides and any degradation product of these compounds or combinations thereof.

20. The method according to claim 1, wherein the biosensor detects only bioavailable compounds.

21. The method according to claim 1, wherein at least one of the host organism has a competence of more then 10⁻⁶, or wherein the host organism displays a rate of integration of about 0.1%.

22. (canceled)

23. The method according to claim 1, wherein the host organism is a gamma bacterium selected from the group consisting of the Acinetobacter species and the Pseudomonas species.

24. The method according to claim 23, wherein the host organism is Acinetobacter baylyi.

25. The method according to claim 1, wherein the means for detecting activation of the promoter or the reporter is a reporter gene selected from the group consisting of β-galactosidase, one or more of the firefly luciferase genes, and the green fluorescent protein (GFP) gene.

26. The method according to claim 1, wherein at least one of the cloned promoter or the cloned gene which encodes the regulatory protein, are derived from an operon used by an organism to metabolise the specific compound.

27. The method according to claim 1, which produces biosensors capable of detecting nanomolar levels of a particular compound.

28. (canceled)

29. A kit for use in detecting a chemical compound in a sample, comprising a biosensor made according to the method of claim 1 and instructions to use the biosensor.

30-32. (canceled)

33. A biosensor produced using the method of claim 1.


SEQUENCE LISTING (15 sequences)

SEQ ID NO: 1 - DNA, 22 bp, Artificial
  primer - salA_flank_for
ccagctgatcagttgtagaatg

SEQ ID NO: 2 - DNA, 40 bp, Artificial
  primer - salA_EB_for
gatgctattttagggagaattccacggatccagtgtaagt

SEQ ID NO: 3 - DNA, 40 bp, Artificial
  primer - salA_EB_rev
acttacactggatccgtggaattctccctaaaatagcatc

SEQ ID NO: 4 - DNA, 22 bp, Artificial
  primer - salA_revH
aacaggttgtattgctgctcgc

SEQ ID NO: 5 - DNA, 23 bp, Artificial
  primer - luxC_rev
gagagtcattcaatattggcagg

SEQ ID NO: 6 - DNA, 20 bp, Artificial
  primer - salAR_rev
gacctgagtatgcccggtag

SEQ ID NO: 7 - DNA, 21 bp, Artificial
  primer - luxE_for
tggtttaccagtagcggcacg

SEQ ID NO: 8 - DNA, 23 bp, Artificial
  primer - xylR1_for
tggatttcagttaatcaattggt

SEQ ID NO: 9 - DNA, 21 bp, Artificial
  primer - xylR_rev
ctatcggcccattgctttcac

SEQ ID NO: 10 - DNA, 28 bp, Artificial
  primer - Sigma54_for
atgaagttatctgttggattgaaagtcg

SEQ ID NO: 11 - DNA, 24 bp, Artificial
  primer - Sigma54_rev
cacgatattttgcaacggttctac

SEQ ID NO: 12 - DNA, 32 bp, Acinetobacter sp.
cgataaagtcatctaccgggcatactcaggtc

SEQ ID NO: 13 - DNA, 28 bp, Artificial
  delta-salR with 4bp deletion and a BglII site\ (see page 46 of description as filed)
cgagatctctaccgggcatactcaggtc

SEQ ID NO: 14 - DNA, 3392 bp, Artificial
  Km gene flanked by salA fragments 1 and 2 (sequence in Figure 8)
nnnnnttnnggtgacntatagaatactcaagctatgcatccaacgcgttgggagctctcccatatggtcgacctgcaggcggccgcactagtgattccagctgatcagttgtagaatgaaaaaataagtgcctgccagaatcaaaaaagttccagccaaaaatagatattgcatcaaaccttggaatgagctcaattcaatcagtcgttcataaaaacgagcatttgtaagacttgctaaccaacaagctagaagcccattaaatgtatttatagataaacttacttttttgaattgtagcaatttaaacatctcaatatcgttcagttatttgaggggtatagctacattaaagagtatgaaaattaagagaaaattagttttcgcttcaatgattcaatttaaatctaaatatcaatgttttaagatcataatgatggatatgaaaacttaaacctataccggataggcaattaagaaagaaatatgatataattgttttaagatattgattttaataaattatttaaaaaattaataactgctttaaaaaactttaaattggatttaattgagtttgcagactaaaaataaaacaataaatcaagttctcaaaggaaatgagtcgtgggtaaaaaaattagtatagccattattggtggtggtatttgcaggggtcgcgcttgcagccaacttatttaagcaaccacatttagaggtttgcttgtatgaagcagcacctcaattttctgaaattgggtgccggagtttcatttggcgcgaatgcggtttgtgccattgagctacttggtttggcttcgcaatataccgcaattgcagatcaagtatctgcgccatttcaggatgtgtggtttcaatggcgaaatggttataacgatgattatttgtcgagttcaatttctcctcaggttggtcagtcttcggtgcatcgtgccgatttcttagatgctattttagggagaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaacctcatacagaaaattcatttactaacgtctggaaagacgacaaaactttagatccggggaattgggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatccagtgtaaatttaataaaaaactcaaatcgattcaggagtacgacacacatattgaattgagttttgaagatggtacatgcgccgaagccgattatgtgattggtgcagatggcatacactcagccacgcgtgattatgtgctgcaaacccatcagtttgctccagtgcgtcctaattttactggaacatgggcttaccgaggcattatcaaagcagcagaatttaggcaagccattgtcgcagccggcctagatgtagaaattgccgacgtaccacaaatgtttttaggccaaaacaagcacattttaacctttccgattcgtcaaggtgaagacattaacattgtggcgtttaagacaaaccctgagcagcgtacgcttccagaacataccccatggacacgtgcggtagacaaacaggaaatgttggacgattttcaggactggagcgaaagctgccgaattttactcggtttgattgagcgtccgaccttgtgggcactgcacgaactggccgaattgccgacttatcaaagccactctggccgcgtcatattaatgggagatgctgcgcacgccatgcttccacatcagggtgccggagcaggacaagggcttgaagatgcactcacgctcaaagtattgtttgagcacactgagctgactgttgaagatttaccgcgagtttctgcaatttatgaacagatccgaaaagaacgcgcctgcaaggttcagcgtacttcgcgtgagtctgggcaaatatatgaacttaactcagcactgtatccaagctttgaagcagtgggtgcacatttgcaaaaccgttttgactggttatggcagcatgatttagcacaagacatgttagccgcgcgagcagcaatacaacctgttaatcccgcggccatggcggcngnagcatgcgnnnngnnnn

SEQ ID NO: 15 - DNA, 2025 bp, Artificial
  salA and salR fragments (sequence in Figure 9)
ctcaaaggaaatgagtcgtgggtaaaaaaattagtatagccattattggtggtggtattgcaggggtcgcgcttgcagccaacttatttaagcaaccacatttagaggtttgcttgtatgaagcagcacctcaattttctgaaattggtgccggaatttcatttggcgcgaatgcggttcgtgccattgagctacttggtttggcttcgcaatataccgcgattgcagatcaagtatctgcgccatttcaggatgtgtggtttcaatggcgaaatggttataacgatgattatttgtcgagttcaatttctcctcaggttggtcagtcttcggtgcatcgtgccgatttcttagatgctattttagggaatattccacagcaccagtgtaagtttaataaaaaactcaaatcgattcaggagtacgacacacatattgaattgagttttgaagatggtacatgcgccgaagccgattatgtgattggtgcagatggcatacactcagccacgcgtgattatgtgctgcaaacccatcagtttgctccagtgcgtcctaattttactggaacatgggcttaccgaggcattattaaagcagcagaatttaggcaagccattgtcgcagccggcctagatgtagaaattgccgacgtaccacaaatgtttttaggccaaaacaagcacattttaacctttccgattcgtcaaggtgaagacattaacattgtggcgtttaagacaaaccctgagcagcgtacgcttccagaacataccccatggacacgtgcggtagacaaacaggaaatgttggacgattttcaggactggagcgaaagctgccgaattttactcggtttgattgagcgtccgaccttgtgggcactgcacgaactggccgaattgccgacttatcaaagccactctggccgcgtcatattaatgggagatgctgcgcacgccatgcttccacatcagggtgccggagcaggacaagggcttgaagatgcactcacgctcaaagtattgtttgagcacactgagctgactgttgaagatttaccgcgagtttctgcaatttatgaacagatccgaaaagaacgcgcctgcaaggttcagcgtacttcgcgtgagtctgggcaaatatatgaacttaactcagcactgtatccaagctttgaagcagtgggtgcacatttgcaaaaccgttttgactggttatggcagcatgatttagcacaagacatgttagccgcgcgagcagcaatacaacctgttgcaacgatttaaacgctaagaattcggatccagagtgttttgaacgacttgtgcctttaaaacaattctattttgaaagagttgaataaaagtgtttatgataggattaaattaaaatcatggaagattctaaaacgtggacttaagcctgatccgtatttttatttgtgtttatgaaaataaaaatatcagtaaagccgctgagattttaaatctgagtcaaccttctgttacctataatttaaatcgattacgtaagcatttaaataatcctttatttgaacgcacgcaatatggtgtggaagcaactaaattatcgcatgaattatatcctgtatttaaagagtcgattttaaaaattgaaatagcagtcgatgaggcattaaattttaatccactcacttcaaataaaacctttcgaattggtttatcagatattggtgaaatttgcttgttaccgacattaatcgaatacttacgagcacatgcaccaaaaataaaaatagaagtagaagagattaaaattgatcaagtcgaaaaatggttaatcgaaggatttattgatgtggctgtttttaatagtacacatttggagtttaagcatcttgaatatgaaactctttttttagagcgatatgttgcactggtcaacatgaatcatccgcgaataagaagtacgctcagttttgatgcgtatttgaatgaatctcatgtggcgataaagtcatctaccgggcatactcaggtc